(19) United States
(12) Patent Application Publication
(10) Pub. No.: US 2005/0074493 A1
(43) Pub. Date: Apr. 7, 2005

(54) Extended release formulations of opioids and method of use thereof

(76) Inventors: Atul M. Mehta, Mahwah, NJ (US); Manish S. Shah, West Caldwell, NJ (US)

Correspondence Address: KENYON & KENYON, One Broadway, New York, NY, 10004, US

(21) Appl. No.: 10/951,978

(22) Filed: Sep. 28, 2004

Related U.S. Application Data
(60) Provisional application No. 60/508,531, filed on Oct. 3, 2003.

(51) Int. Cl. A61K 31/485; A61K 9/54; A61K 9/20; A61K 9/26

(52) U.S. Cl. 424/469; 514/282

(57) ABSTRACT

Extended release formulations for the delivery of opioid agonists, including oxycodone, are provided which exhibit low peak to trough plasma concentration fluctuations and sufficiently high plasma concentrations over an extended period of time to provide a once a day dosage administration, wherein the formulations provide pain relief for up to 24 hours The extended release formulations may be customized to achieve the desired plasma concentration profile, e.g. two or more different extended release drug-loaded pellets or granules may be combined in a formulation. Additional formulations include combinations of drug loaded and extended release opioid agonists-loaded pellets or granules. Other formulations include a combination of an opioid agonist and an opioid antagonist, as well as a combination of an opioid agonist and a NSAID.

### FIELD OF THE INVENTION

This invention relates to oral formulations of pain medications, specifically opioid agonists formulations, which have excellent sustained release, i.e., extended release properties, thereby reducing the number of daily opioid agonist dosage administrations to patients to once a day. In particular, this invention relates to oxycodone formulations, which provide reduced peak to trough plasma concentrations of oxycodone, provide pain relief for up to 24 hours, and have potential to reduce incidence of breakthrough pain.

### BACKGROUND OF THE INVENTION

There exists a need in the art of pain management and pain therapy for a dosage form of drugs used in pain management, such as opioid agonists, including oxycodone, codeine, morphine, and the like which releases the drug over extended periods of time, and as a result provides prolonged therapeutic effect. It is also necessary that drugs including the aforementioned opioid agonists provide reduced peak to trough plasma concentrations and minimum plasma concentrations such that breakthrough pain is reduced in patients.

As is well known, the maximum time effectiveness in many pharmaceutical preparations containing a drug is only a few hours because of biological modification and elimination of the medication in the body. Consequently, repeated doses must be taken at frequent intervals to obtain long term therapeutic levels of drugs. After high initial peak concentrations, the level of drug in the blood stream continually decreases due to biological elimination, so there is little or no therapeutic effect at the end of the period between doses. As a result, the therapeutic effect fluctuates between doses corresponding to the peaks and valleys in the level of drug in blood.

The activity of oxycodone and other opioid agonists in humans is directly related to its blood or plasma concentration. Likewise, there is a general relationship between increasing oxycodone plasma concentration and increasing frequency of dose-related adverse side effects, e.g. nausea, vomiting, somnolescence and respiratory depression. For illnesses which require continuous and constant control of moderate to severe pain, pain management drugs, e.g. oxycodone, generally require administration about every 6 hours for immediate-release dosage forms and about every 12 hours for controlled-release dosage forms. In addition to the above-mentioned side effects, a rapid increase of an opioid agonist in the blood and high plasma concentrations thereof may also cause undesired gastrointestinal and other smooth muscle effects, e.g. constipation. Accordingly, the analgesic treatment of diseases in which moderate to severe pain is managed by opioid agonists such as oxycodone, requires providing an adequate high blood plasma level of the opioid agonist for an extended period of time, and limiting the adverse side effects, some of which may potentially be fatal. Moreover, narcotics such as opioid agonists, including e.g., oxycodone, pose a risk of abuse, therefore, a need also exists for abuse-prevention dose formulations, i.e., abuse-resistant opioid agonist formulations comprising opioid antagonists. Since the treatment of some diseases requires not only pain management but also management of inflammation, dosage formulations comprising an opioid agonist and a non-steroidal anti-inflammatory drug (NSAID) are also needed.

At present pharmaceutical pain management drug formulations of opioid agonists are commercially available in immediate and twice a day sustained-release forms. For example, oxycodone, an opioid agonist drug that is utilized in the treatment and management of moderate to severe pain, is sold commercially in immediate-release oral capsule (OxyIR®), immediate-release oral concentrate solution (Oxyfast®), and twice a day controlled-release tablet (OxyContin®) dosage forms. The commercially available immediate-release oxycodone 5 mg formulations achieve peak plasma levels at 1.6 hours after administration, while controlled-release OxyContin® tablets reach maximum plasma concentration from 2.1 to 3.2 hours after administration, depending upon the dose administered (Purdue Pharma L.P. product information).

U.S. Pat. Nos. 4,861,598, and 4,970,075 to Oshlack describes extended action controlled release pharmaceutical compositions for oral administration, which may include as a pharmaceutically active agent oxycodone. The pharmaceutical compositions described therein contain a higher aliphatic alcohol of 10-18 carbon atoms and a pharmaceutically acceptable acrylic resin, said acrylic resin being in an amount of about 10-60% by weight of the weight of said higher aliphatic alcohol plus said acrylic resin. In an exemplified formulation, 43% of oxycodone is released from a tablet formulation in one hour and 100% in 5 hours; in a second exemplified formulation, 16% of oxycodone is released from a tablet formulation in one hour and 100% in 9 hours. These formulations do not provide for release of the oxycodone over an extended period of time.

U.S. Pat. No. 5,266,331 to Oshlack et al. describes a controlled release formulation comprising an analgesically effective amount of oxycodone or a salt thereof in a matrix. The formulation described provides a dissolution rate in vitro of the dosage form, when measured by the USP Paddle Method at 100 rpm at 900 ml aqueous buffer (pH between 1.6 and 7.2) at 37° C. of between 12.5% and 42.5% (by wt) oxycodone released after 1 hour, between 25% and 55% (by wt) oxycodone released after 2 hours, between 45% and 75% (by wt) oxycodone released after 4 hours and between 55% and 85% (by wt) oxycodone released after 6 hours. The formulations describe the peak plasma level of oxycodone obtained in vivo occurs between 2 and 4 hours after administration of the dosage form. The described formulations provide a high initial release of drug and rapidly reach peak plasma levels.

U.S. Pat. Nos. 5,508,042, 5,549,912, and 5,656,295 to Oshlack et al. describe formulations of oxycodone which provide a mean maximum plasma concentration (Cₘₐₓ) at about 2 hours to about 4.5 hours. The described formulations rapidly attain peak plasma levels. These formulations are therefore required to be given 12 hours apart.

U.S. Pat. Nos. 5,958,459 and 6,103,261 to Chasin et al. and U.S. Pat. No. 6,143,322 to Sackler et al. describe opioid formulations which may include oxycodone; the formulations described provide a dissolution rate in vitro of the dosage form, when measured by the USP Paddle Method at 100 rpm at 900 ml aqueous buffer (pH between 1.6 and 7.2) at 37° C. of between 12.5% and 42.5% (by wt) after 1 hour (U.S. Pat. No. 6,103,261) and between 16.8% and 42.5% (by wt) released after 1 hour (U.S. Pat. No. 5,958,459 and U.S. Pat. No. 6,143,322). The formulations describe the peak plasma level of opioid obtained in vivo occurs between 2 to 8 hours (U.S. Pat. No. 5,958,459 and U.S. Pat. No. 6,143,322) and between 4 to 8 hours (U.S. Pat. No. 6,103,261) after administration of the dosage form.

U.S. Pat. Nos. 5,500,227 and 6,387,404 to Oshlack et al. describe sustained release tablets for oral administration which comprise an immediate release tablet core including an insoluble therapeutically active agent.

U.S. Pat. No. 5,478,577 describes methods for providing effective pain management in humans for a time period of about 24 hours with oral dosage forms of an opioid analgesic which provides large peak to trough fluctuations in opioid levels even after repeated dosing.

Thus, there is a need for an improved form of extended (i.e. sustained and controlled) release drugs for pain management, specifically opioid agonists, e.g. oxycodone, to reduce the peak to trough fluctuations and yet provide reduced dosing requirements, i.e. once a day administration. It is further desirable to achieve sustained plasma concentrations of the drug such that it minimizes a need for rescue dose generally given for breakthrough pain.

There is also a pressing need for a dosage form that can provide an extended delivery of a pain management therapeutic, such as oxycodone, in an effective dose of the therapeutic agent at a controlled delivery of the drug, thereby avoiding a rapid or sudden increase of the drug in the blood while still providing a sustained controlled drug release over a longer period of time, i.e. up to 24 hours, such that pain relief is provided for up to 24 hours, than is presently provided by commercially available formulations of opioid agonists, in particular oxycodone.

There is also a pressing need for such extended release opiod formulations to be rendered abuse resistant by combining an opioid formulation with an antagonist formulation.

It will be appreciated by those versed in the medical arts, that a novel and unique dosage form which provides both extended release of opioids and NSAIDs for effective therapeutic analgesic effect or relief from pain for up to about 24 hours is also a desirable contribution to the medical arts.

### SUMMARY OF THE INVENTION

The present invention provides various dosage formulations of opioid agonists, preferably oxycodone, including: a formulation comprising at least one extended release (ER) opioid-loaded pellet or granule; a formulation comprising at least two different extended release opioid-loaded pellets or granules; a formulation comprising at least three different extended release opioid-loaded pellets or granules; a formulation comprising at least one opioid-loaded pellet or granule and at least one extended release opioid-loaded pellet or granule; a formulation comprising at least one opioid-loaded pellet or granule and at least two extended release opioid-loaded pellets or granules, and a formulation comprising at least one opioid-loaded pellet or granule and at least three extended release opioid-loaded pellets or granules. The present invention also provides dosage formulations of opioid agonists in formulations as described above, wherein each of the formulations may further comprise at least one opioid antagonist coated pellet or opioid antagonist-loaded granule; at least one NSAID coated pellet or NSAID-loaded granule; or at least one opioid antagonist coated pellet or opioid antagonist-loaded granule and at least one NSAID coated pellet or NSAID-loaded granule.

The present invention provides dosage forms for the oral delivery of a pain management drug comprising biologically inert pellets coated with a layer of drug, specifically an opioid agonist, e.g., oxycodone, or a pharmaceutically acceptable salt thereof. In alternate dosage forms for oral delivery of opioid agonists, the opioid agonist is formulated as an opioid-loaded granule. In exemplary embodiments of the present invention, the dosage forms further comprise an extended-release layer comprising a water-insoluble polymer. In further exemplary embodiments of the present invention, the dosage form may include a combination of at least one biologically inert pellet coated with a layer of drug and at least one biologically inert pellet coated with an inner layer of drug and said drug coated pellet or granule further comprising an extended-release layer comprising a water-insoluble polymer. In additional exemplary embodiments of the present invention, the dosage form may comprise a plurality of pellets or granules, said plurality comprising at least one biologically inert pellet coated with a layer of drug or a drug-loaded granule and at least two biologically inert pellets or drug-loaded granules, wherein each of the at least two biologically inert pellets or drug-loaded granules is coated with a layer of drug and said drug coated pellet or drug-loaded granule further comprises an extended-release layer comprising a water-insoluble polymer, wherein the extended-release layer provides a different release rate of the drug for each of the at least two drug coated pellets or drug-loaded granules.

In a further exemplary embodiment, each of the aforementioned dosage forms may also further comprise a coated pellet or drug-loaded granule or a plurality of coated pellets or drug-loaded granules, wherein a pellet is coated or granule is loaded with (a) at least one opioid antagonist, (b) at least one pain management drug, or (c) at least one non-steroidal anti-inflammatory drug (NSAID), wherein a plurality of coated pellets or drug-loaded granules comprises a first pellet coated with or granule loaded with at least one pain management drug and a second pellet coated with or granule loaded with at least one NSAID; a first pellet coated with or granule loaded with at least one opioid antagonist and a second pellet coated with or granule loaded with at least one pain management drug; a first pellet coated with or granule loaded with at least one opioid antagonist and a second pellet coated with or granule loaded with at least one non-steroidal anti-inflammatory drug (NSAID); or a first pellet coated with or granule loaded with at least one opioid antagonist, a second pellet coated with or granule loaded with at least one pain management drug and a third pellet coated with or granule loaded with at least one non-steroidal anti-inflammatory drug (NSAID).

In another exemplary embodiment, the present invention provides an oral dosage form comprising a biologically inert pellet, wherein the biologically inert pellet is coated with an opioid-agonist layer, wherein the opioid-agonist layer comprises an amount of an opioid agonist or salt thereof, which is further combined in an oral dosage form with opioid-loaded pellets or opioid-loaded granules or pharmaceutically acceptable salt thereof, wherein the opioid agonist layer (or opioid-loaded granule) is coated with an extended-release layer, so as to thereby provide a sustained drug release for up to about 24 hours. In preferred exemplary embodiments, the oral dosage form provides pain relief for up to 24 hours. In a further preferred embodiment, the oral dosage form is administered once a day.

In an exemplary embodiment, this invention provides an oral dosage form comprising: a biologically inert pellet, wherein the biologically inert pellet is coated with an opioid-agonist layer, wherein the opioid-agonist layer comprises an amount of an opioid agonist or pharmaceutically acceptable salt thereof, and an extended-release layer coated on the opioid agonist layer, wherein the extended-release layer comprises a water-insoluble polymer; wherein the dissolution rate in vitro of the dosage form, when measured by the USP Paddle method of 100 rpm in 900 ml of deionized water provides an extended release for up to 24 hours of the opioid agonist or pharmaceutically acceptable salt thereof when the oral dosage is orally administered to a human being. In preferred exemplary embodiments, the oral dosage form provides pain relief for up to 24 hours. In a further preferred embodiment, the oral dosage form is administered once a day. These ER pellets may then be incorporated into capsules or tablets to provide the desired in vivo release profiles.

In a further exemplary embodiment, this invention provides an oral dosage form including: a first pellet comprising: a biologically inert pellet, wherein the biologically inert pellet is coated with an opioid-agonist layer, wherein the opioid-agonist layer comprises a therapeutically effective amount of an opioid agonist; and a second pellet comprising: a biologically inert pellet; an opioid-agonist layer coated on the biologically inert pellet, wherein the opioid-agonist layer comprises a therapeutically effective amount of an opioid agonist; and an extended-release layer coated on the opioid agonist layer, wherein the extended-release layer comprises a water-insoluble polymer wherein the oral dosage form provides an extended release for up to about 24 hours of the opioid agonist or pharmaceutically acceptable salt thereof when the dosage is orally administered to a human being. In preferred exemplary embodiments, the oral dosage form provides pain relief for up to 24 hours. In a further preferred embodiment, the oral dosage form is administered once a day. Any combination of drug layered pellets (also referred to herein as drug loaded pellets) and ER pellets can be combined, i.e. one drug loaded pellet plus one ER pellet, one drug loaded pellet plus two ER pellets, or one drug loaded pellet and three ER pellets. Exemplary embodiments of drug-loaded, e.g. oxycodone-loaded, pellets are provided in Examples 1 and 2, but are not limited thereto. The various combinations of ER pellets, whether two ER pellets or three ER pellets may be selected from Examples 3 to 16. Exemplary embodiments of oral dosages forms formulated using various combinations of these drug-loaded pellets and ER pellets are provided in Examples 17 to 32, but are not limited thereto.

In another exemplary embodiment, this invention provides an oral dosage form including: a first pellet comprising: a biologically inert pellet, wherein the biologically inert pellet is coated with an opioid-agonist layer, wherein the opioid-agonist layer comprises a therapeutically effective amount of an opioid agonist; and an extended-release layer coated on the opioid agonist layer, wherein the extended-release layer comprises a water-insoluble polymer; and a second pellet comprising: a biologically inert pellet; an opioid-agonist layer coated on the biologically inert pellet, wherein the opioid-agonist layer comprises a therapeutically effective amount of an opioid agonist and wherein the amount of the opioid agonist is the same as the amount of opioid agonist coated on the biologically inert pellet of the first pellet; and an extended-release layer coated on the opioid agonist layer, wherein the extended-release layer comprises a water-insoluble polymer in an amount different than the amount of water-insoluble polymer coated on the opioid agonist layer of the first pellet such that the first pellet and the second pellet each have a different rate of release of the opioid-agonist when orally administered to a human being; wherein the oral dosage form provides an extended release of the therapeutically effective amount of the opioid agonist when the dosage is orally administered to a human being.

The various combinations of ER pellets, whether two ER pellets or three ER pellets may be selected from Examples 3 to 16. Exemplary embodiments of drug-loaded, e.g. oxycodone-loaded, pellets are provided in Examples 1 and 2, but are not limited thereto. Exemplary embodiments of oral dosages forms formulated using various combinations of these drug-loaded pellets and ER pellets are provided in Examples 17 to 32, but are not limited thereto. In preferred exemplary embodiments, the oral dosage form provides pain relief for up to 24 hours. In a further preferred embodiment, the oral dosage form is administered once a day.

In another exemplary embodiment, the present invention provides an oral dosage form including a first pellet comprising: a biologically inert pellet, wherein the biologically inert pellet is coated with an opioid-agonist layer, wherein the opioid-agonist layer comprises a an opioid agonist or a pharmaceutically acceptable salt thereof; and an extended-release coated on the opioid agonist layer, wherein the extended-release layer comprises a water-insoluble polymer; a second pellet comprising: a biologically inert pellet; an opioid-agonist layer coated on the biologically inert pellet, wherein the opioid-agonist layer comprises a therapeutically effective amount of an opioid agonist and wherein the amount of the opioid agonist is the same as the amount of opioid agonist coated on the biologically inert pellet of the first pellet; and an extended-release layer coated on the opioid agonist layer, wherein the extended-release layer comprises a water-insoluble polymer in an amount different than the amount of water-insoluble polymer coated on the opioid agonist layer of the first pellet; and a third pellet comprising: a biologically inert pellet; an opioid-agonist layer coated on the biologically inert pellet, wherein the opioid-agonist layer comprises an opioid agonist or a pharmaceutically acceptable salt thereof and wherein the amount of the opioid agonist is the same as the amount of opioid agonist coated on the biologically inert pellet of the first pellet; a extended-release layer coated on the opioid agonist layer, wherein the extended-release layer comprises a water-insoluble polymer in an amount different than the amount of water-insoluble polymer coated on the opioid agonist layer of the first pellet or of the second pellet; wherein the release rate of the first pellet, the release rate of the second pellet, and the release rate of the third pellet are each different, wherein the oral dosage form provides an extended release of the therapeutically effective amount of the opioid agonist when the oral dosage is orally administered to a human being. The various combinations of ER pellets, whether two ER pellets or three ER pellets may be selected from Examples 3 to 16. Exemplary embodiments of drug-loaded, e.g. oxycodone-loaded, pellets are provided in Examples 1 and 2, but are not limited thereto. Exemplary embodiments of oral dosages forms formulated using various combinations of these drug-loaded pellets and ER pellets are provided in Examples 17 to 32, but are not limited thereto. In preferred exemplary embodiments, the oral dosage form provides pain relief for up to 24 hours. In a further preferred embodiment, the oral dosage form is administered once a day.

In another exemplary embodiment of the present invention, there is provided a method of preparing an extended-release oral dosage form of oxycodone, said method comprising: coating a biologically inert pellet (or drug loaded granule) with a dose of oxycodone or a pharmaceutically acceptable salt thereof, wherein the oxycodone layer comprises an amount of oxycodone or a pharmaceutically acceptable salt thereof, to form an oxycodone loaded pellet or granule; and coating the oxycodone loaded pellet or granule with an extended-release layer, wherein the extended-release layer comprises a water-insoluble polymer; wherein the oral dosage form provides an extended drug release of up to 24 hours of the oxycodone when the dosage is orally administered to a human being. In preferred exemplary embodiments, the oral dosage form provides pain relief for up to 24 hours. In a further preferred embodiment, the oral dosage form is administered once a day.

In another exemplary embodiment, the present invention provides a method of treating pain in a subject in need thereof, said method comprising orally administering to the subject an oxycodone oral dosage form, as described above, said oral dosage form comprising: at least one pellet or granule comprising: a biologically inert pellet (or an oxycodone-loaded granule); an oxycodone layer coated on the biologically inert pellet (or an oxycodone-loaded granule), wherein the oxycodone layer (or the oxycodone component of the granule) comprises a therapeutically effective amount of oxycodone, to form an oxycodone loaded pellet or granule; and an extended release layer coated on the oxycodone layer (or oxycodone-loaded granule), wherein the extended release comprises a water-insoluble polymer; wherein the oral dosage form provides an extended release of up to 24 hours of the oxycodone when the dosage form is orally administered to a human being. In preferred exemplary embodiments, the oral dosage form provides pain relief for up to 24 hours. In a further preferred embodiment, the oral dosage form is administered once a day. The oral dosage of the oxycodone or pharmaceutically acceptable salt thereof may have any of the suitable in vitro dissolution rates. Preferably, the in vitro dissolution rate of the formulation is chosen such that it provides reduced peak to trough plasma concentrations, provides pain relief for up to 24 hours, and reduces incidence of breakthrough pain, thereby minimizing the need for “rescue” doses of the opioid agonist, i.e. additional administration of opioid dosages within the extended time period of opioid release.

### BRIEF DESCRIPTION OF THE DRAWINGS

FIG. 1 is a semi-log graph of the mean plasma concentrations under fasting conditions of two oxycodone formulations. Examples 20 and 21, and two currently marketed oxycodone formulations, Oxycontin® and Oxyfast®.

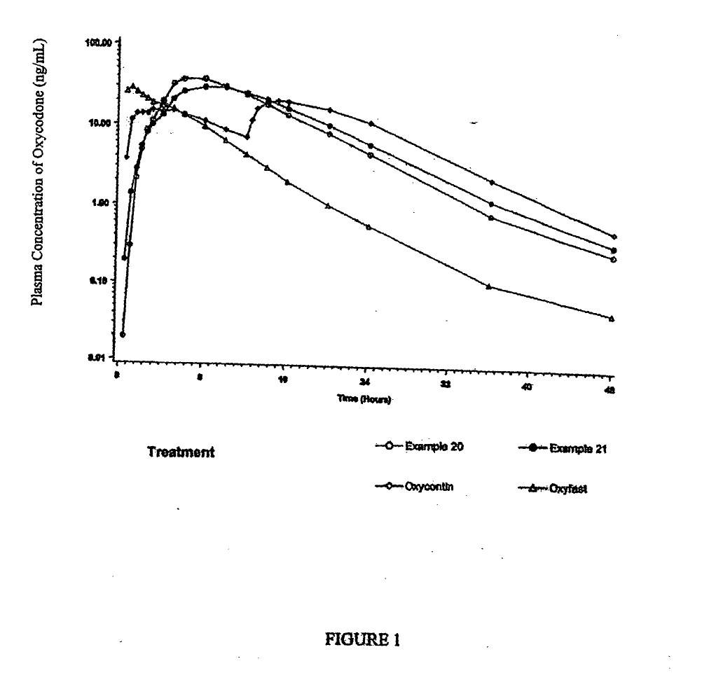

FIG. 2 graphically depicts the in-vitro percent of oxycodone dissolved over 24 hours for two oxycodone formulations, Examples 20 and 21.

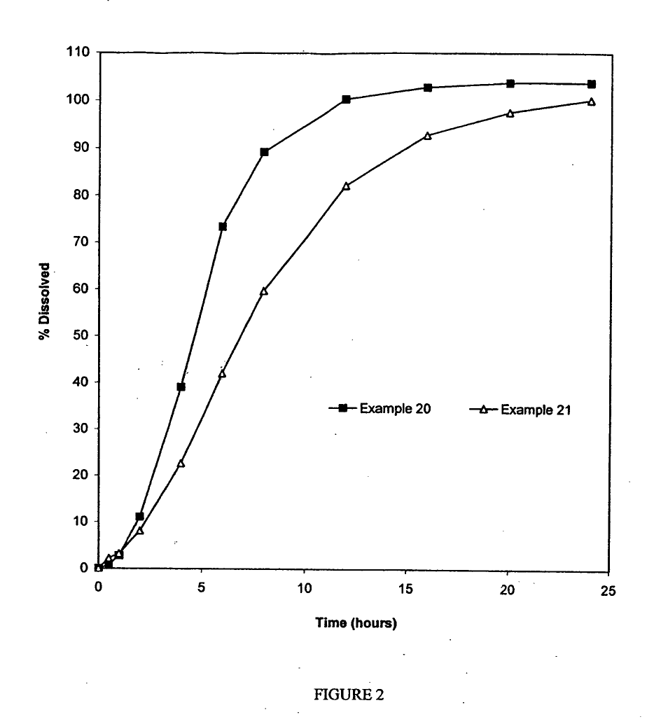

FIG. 3 depicts the in vitro-in vivo correlation obtained for oxycodone formulations, Examples 20 and 21, whose in vitro dissolution profiles are shown in FIG. 2.

FIG. 4A shows a plot of mean plasma oxycodone concentration versus time under fasting condition which represents a simulated desired in-vivo profile. The oxycodone level does not fall below 10 ng/ml during at least a 24 hour time interval.

FIG. 4B shows the necessary percentage dissolution profile in order to achieve the desired in-vivo profile.

FIG. 4C is a graph of simulated plasma concentrations at steady state of two oxycodone formulations: currently marketed Oxycontin® and the formulations according to the present invention.

### DETAILED DESCRIPTION OF THE INVENTION

This invention, as disclosed and described herein, provides novel extended, i.e. sustained and controlled release formulations of drugs used for management of pain when administered orally. As used herein the phrase “pain management drug” includes any narcotic or non-narcotic pain reliever (analgesic), which is used in the management of mild to severe pain and/or fever. Such pain management drugs include but are not limited to, for example, oxycodone, codeine, morphine, hydromorphine, anilevidine, merperidine, methadone, levorphanol, pentazocine, propoxyphene, alfentanil, allyprodine, alphaprodine, anileridine, benzylmorphine, bezitramide, buprenorphine, butorphanol, clonitazene, cyclazocine, desomorphine, dextromoramide, dezocine, diampromide, dihydrocodeine, dihydromorphine, dimenoxadol, dimepheptanol, dimethylthiambutene, dioxaphetyl butyrate, dipipanone, eptazocine, ethoheptazine, ethylmethylthiambutene, ethylmorphine, etonitazene, fentanyl, heroin, hydrocodone, hydromorphone, hydroxypethidine, isomethadone, ketobemidone, levallorphan, levophenacylmorphan, lofentanil, meptazinol, metazocine, metopon, myrophine, nalbuphine, narceine, nicomorphine, norlevorphanol, normethadone, nalorphine, normorphine, norpipanone, opium, oxymorphone, papavretum, phenadoxone, phenomorphan, phenazocine, phenoperidine, piminodine, piritramide, propheptazine, promedol, properidine, propiram, sufentanil, tramadol, tilidine, and the like, or salts thereof, as well as combinations thereof.

In multiple drug component formulations, wherein the second drug is an anti-inflammatory drug, such drugs include but are not limited to NSAIDs, such as diclofenac, flufenamic acid, flurbiprofen, ibuprofen, indomethacin, ketoprofen, naproxen, phenylbutazone, sulindac, piroxicam, salicylic acid, acetylsalicylic acid, celecoxib, etodolac, fenoprofen, ketoralac, oxaprozin, nabumetone, tolmetin, and rofecoxib and the like. It is also understood that some of the NSAIDs may also be pain management drugs and/or antipyretic agents, such as for example, acetylsalicylic acid, ketoprofen and the like.

The formulations of the present invention may also contain a combination of a pain management drug, e.g. opioid agonist and an opioid antagonist. An opioid antagonist may be selected from, but is not limited to, the group consisting of naltrexone, naloxone, nalmephene, and nalorphine. This invention also provides pain management drug formulations comprising an opioid antagonist and/or a NSAID, wherein such formulations optimize pain management drug and/or inflammation and further provide opioid-abuse resistant dosage forms. Each of the aforementioned dosage forms provides a formulation which exhibits a predictable time to reach maximum blood concentration levels of active agent and provides less variation in maximum blood concentrations than prior art formulations.

The present invention provides novel formulations of pain management drugs, specifically opioid agonists, preferably oxycodone, characterized by a release profile that results in enhanced pharmacokinetic performance. These formulations also afford excellent bioavailability while avoiding high plasma concentration peaks to minimize high peak to trough fluctuations.

The extended release pain management drug formulations of the invention exhibit a time-dependent release of drug, rather than a pH-dependent release exhibited by prior art formulations that depend on an outer pH-sensitive enteric coating to delay release.

In one embodiment of the extended release formulation of the present invention, the one or more layers of pain management drug(s), may be coated with an extended-release layer rate and may optionally be seal coated. In accordance with the present invention, any pain management drug(s) or its pharmaceutically acceptable salt, and preferably oxycodone or any pharmaceutically acceptable salt oxycodone may be used as the drug. For example, such salts may include the sodium or potassium salts. It is preferred however, that the hydrochloride salt of oxycodone be used. Any pain management drug that is an opioid agonist may be used in the present formulations or combinations thereof.

In another exemplary embodiment of the present invention, any opioid agonist may be combined with an opioid antagonist, a NSAID, or another pain management drug, and/or combinations thereof, to form a two-component or multiple-component dosage formulation. In a preferred exemplary embodiment the pain management drug, i.e. opioid agonist, is oxycodone. In multiple component formulations, wherein the second therapeutic agent/drug is an opioid antagonist, preferably, the opioid antagonist is naltrexone. Naltrexone formulations preferably include, but are not limited to, the formulations of copending patent application U.S. Ser. No. 10/409,992, filed Apr. 8, 2003, the entire contents of which are hereby incorporated by reference in their entirety, specifically naltrexone dose formulations. In multiple component formulations, wherein the second drug is a NSAID, preferably the NSAID is diclofenac. Diclofenac formulations preferably include, but are not limited to, the formulations of copending patent application U.S. Ser. No. 09/659,903, filed Oct. 26, 2000 and PCT International application no. PCT/US01/32446, filed Oct. 18, 2001 and published as WO 02/034240 on May 2, 2002, the entire contents of which are hereby incorporated by reference in their entirety, specifically diclofenac dose formulations.

As used herein a “pellet” is defined as a biologically inert pellet, which may be coated/layered with a drug, e.g. an opioid agonist, which may then further be coated with an extended release layer. In an example embodiment, a pellet may be a sugar sphere. Such drug-coated pellets may then be placed into a capsule in the desired dosage amount. As used herein a “granule” is defined as a mixture of a drug, e.g. an opioid agonist, and biologically inactive excipients which are formulated into a dosage form. A granule may be coated with an extended release layer. As used herein “extended release layer” is defined as a protective coating of a drug-loaded pellet or granule which provides release of the drug in a sustained and controlled manner over a time period of up to 24 hours.

Many types of pellets that are suitable for use in forming the core of the formulations of the present invention are commercially available from a number of pharmaceutical supply companies; for example, non-pareils, sugar and/or starch-based biologically inert pellets. Non-pareil pellets of particle size 25 to 30 mesh are particularly preferred, although any non-pareil pellet of mesh size within the range of 14 mesh to 60 mesh are also preferred for use in this invention. Alternatively granules can be prepared by conventional techniques known in the art containing the drug. In further embodiments the oral dose formulation comprising either one opioid agonist, e.g. oxycodone, or oxycodone and at least one second therapeutic agent, e.g. an opioid antagonist, may also be made in a tablet form by using standard known techniques in the art.

Suitable binder agents for use in the drug layer of the formulations of the present invention include, for example, hydroxypropylmethyl cellulose, hydroxyethyl cellulose, hydroxypropyl cellulose, methyl cellulose, polyvinyl pyrrolidone and the like. Preferably, hydroxypropylmethyl cellulose is used in the practice of the present invention. Preferably, the binder agent is dissolved in water (or any suitable solvent) to form a 5% to 30% (w/w) solution, preferably a 7% to 25% (w/w) solution and most preferably, an approximately 10% (w/w) solution. The solution of binder agent is admixed with a solution or suspension of the pain management drug, and then applied onto the pellets by conventional film (or spray) techniques. In formulations comprising at least one an additional therapeutic agent(s), such therapeutic agent(s) may be combined with a binder and applied onto separate pellets. In preferred exemplary embodiments of oral formulations provided, preferably, the additional therapeutic agent may be a NSAID. Preferably, the NSAID is diclofenac. In another preferred exemplary embodiments of oral formulations provided, another agent such as an opioid antagonist may be added to the formulation. Preferably, the opioid antagonist is naltrexone.

For example the drug/binding agent solution may be applied to the pellets by spraying the solution or suspension onto the pellets using a fluid processor. The binder agent may constitute about 2-30%, preferably about 4-25%, and most preferably about 5-20% of the total weight of drug in the formulation.

The drug layer of the modified release formulations of the present invention may include one or more pharmaceutically acceptable excipients in addition to the pharmacologically active agent(s) and binder agent. Pharmaceutically acceptable excipients which may be employed are well known to those of skill in the art and include any conventional pharmaceutically acceptable excipient, such as an antifoam agent, which is added to aid the formulation process. The drug layer may include a suitable carrier or diluent, and may optionally contain a surfactant. In another embodiment of the invention, the drug layer may be coated with a sealing layer.

The optional sealing layer contains a water soluble polymer, which may be the same or different from the binder agent present in the drug layer. For example, the sealing agent may include a water soluble polymer such as hydroxypropylmethyl cellulose, hydroxyethyl cellulose, hydroxypropyl cellulose, methyl cellulose, polyvinylpyrrolidone and the like. Preferably, hydroxypropylmethyl cellulose, and most preferably, hydroxypropyl-methyl cellulose-E-6 is used in the sealing layer.

The total amount of optional sealing layer contained in the pharmaceutical loaded pellets may be varied. The sealing layer may constitute from about 0.5 to 5% of the total weight of the formulation.

The pain management drug, e.g., opioid agonist or pharmaceutically acceptable salt thereof loaded pellets may in one embodiment be substantially enveloped with a layer of a water-insoluble polymer referred to herein as a “extended-release layer.” The extended-release layer sufficiently protects the integrity of the drug-loaded pellets for the desired period of time and provides an extended, i.e. sustained or controlled, release rate of the oral dosage form for up to 24 hours in which a steady state of the drug is maintained in the plasma without having large peak to trough plasma concentrations, thereby providing pain relief for up to 24 hours. Once the desired, pre-delivery time has elapsed the drug is released at a rate that provides decreased variation in plasma concentrations of the pain management drug, e.g. an opioid agonist, preferably oxycodone, and decreased peak to trough blood plasma concentration so as to provide a sustained release for a predetermined amount of time, preferably for up to 24 hours, and to provide pain relief for up to 24 hours.

The extended release layer may be comprised of ethyl cellulose, a copolymer of acrylic and methylacrylic acid esters, which is physiologically acceptable, water insoluble, and permeable to the release of the pain management drug, preferably an opioid agonist, most preferably oxycodone, or a pharmaceutically acceptable salt thereof, such as Eudragit RL 30 D, Eudragit RS 30D, or a poly(meth)acrylate polymer, such as Eudragit NE 30 D or Eudragit NE40D, or a combination thereof. Most preferably, the poly(meth)acrylate polymer, Eudragit NE 30 D, is used in formulating the controlled release coating. Eudragit NE 30 D, Eudragit NE40D, Eudragit RS 30 D and Eudragit RL 30 D polymers are available from Rhom Pharma, D-6108 Weiterstadt 1, Dr. Otto-Rohm-Str. 2-4, Germany. Eudragit NE 30 D and Eudragit 40D are pH independent polymers available as 30% or 40% aqueous dispersions, respectively. Eudragit RS and Eudragit RL 30 D are available as aqueous dispersions containing 30% dry substances. The NE30D solids in the rate controlling layer generally constitutes about 2%-50% of the total weight of the solids content of the present formulations, preferably about 4%-30%, and most preferably about 5%-20% of the total weight of the solids content of the present formulations. In a preferred embodiment of the invention the binder agent used in the drug layer is hydroxypropylmethyl cellulose and the extended-release layer is Eudragit NE 30 D.

In a preferred embodiment, the extended release layer may contain in addition to a water-insoluble polymer an amount of a lubricant, such as for example, calcium stearate, magnesium stearate, zinc stearate, stearic acid, talc or a combination thereof to form the rate controlling layer. In particular, it is preferred that the extended-release layer contains an amount of calcium stearate sufficient or other lubricant to provide extended release of oxycodone sufficient for up to about 24 hours after administration of the formulation and thereby provide pain relief for up to 24 hours. In a most preferred embodiment the extended release layer contains calcium stearate admixed with the water insoluble polymer, which is preferably Eudragit NE 30 D. The lubricant functions to prevent agglomeration of the coated pellets during processing and also helps to delay release of the pharmaceutical agent from the coated pellets.

In a preferred embodiment, the final, dried extended-release layer contains about 0.5-15% calcium stearate and/or other lubricant agent(s), and more preferably about 1%-10%, and most preferably about 1.5%-7.5% lubricant agent based on the total weight of solids content of the total formulation.

Optionally, the extended-release layer may contain an amount of a water soluble polymer in addition to the water insoluble polymer.

In another embodiment of the invention the extended-release layer may be coated with an enteric coating polymer, which may optionally contain a plasticizer. A preferred enteric coating polymer is Eudragit L 30D. Suitable plasticizers for inclusion in the enteric layer include, for example, triethyl citrate, polyethylene glycol, dibutyl phthalate, diethylphthalate and triacetin. The optional enteric coating, which is pH dependant and resistant to gastric fluids may comprise from about 3-10%, preferably about 4-6% of the total weight of solids content of the formulation. The optional enteric coating and/or the extended-release layer may also be coated with one or more layers of a sealant or a binding agent.

The pain management drug, e.g., an opioid agonist, preferably an oxycodone layer, an optional sealing layer, an extended-release layer and an optional enteric coating may each further comprise diluents, carriers, fillers and other pharmaceutical additives which may or may not effect the rate of release of active agent(s) from the single pellet. For example, the extended-release layer preferably contains a lubricant agent(s) and the drug layer may optionally contain surfactants. The pellet layers may further contain pharmaceutically acceptable excipients such as anti-adherents, pharmaceutically acceptable pigment such as, titanium dioxide, iron oxide and various color pigments including vegetable dyes, and the like.

Preferably, the pharmaceutical loaded pellets of the invention provide in total a potency of approximately 33% (w/w) based upon the total weight of the layered pellets, although the potency can be adjusted as desired. For example, when the pharmaceutical agent included in the layering is oxycodone, it is preferred that the layered pellet be formulated at about 10 to about 60% potency (w/w). However, the skilled practitioner can formulate the modified release formulations of the invention to have any desired total potency of opioid agonist.

Depending upon the specific opioid agonist present in the extended release formulations and any additional therapeutic agents such as an opioid antagonist or a NSAID, the dose of drug(s) will vary. For example, oxycodone is generally recommended for use in the treatment of mild to severe pain at a dose of from 10 to 400 mg per dose, depending on the form of administration and frequency. Codeine is routinely recommended at a dosage of 10 mg to 60 mg depending on the severity of pain, to be taken every 4 to 6 hours as needed. The specific dose amount may vary from drug to drug by a factor of 30. The dosage amount of any of the pain management drugs, specifically oxycodone, and any additional therapeutic agent such as a NSAID or an opioid antagonist used in the formulations of the present invention can readily be determined by those skilled in the art. Of course, those skilled in the art will recognize that the dosage amount can be varied according to a patient's needs, e.g., adult versus child, other medications being administered, opioid tolerance, etc.

The extended release formulation, as disclosed herein, permits the extended release of the opioid agonist, specifically oxycodone, or a pharmaceutically acceptable salt thereof, in a manner to provide and maintain a reduced variability of plasma drug concentrations and timing of maximum plasma concentrations in comparison to that attained with prior art oxycodone formulations. In preferred formulations of the present invention, the drug is released in a sustained manner to provide drug plasma levels for up to about 24 hours in therapeutically effective amounts, i.e. analgesically effective, wherein such plasma levels are adequate to provide pain relief for up to 24 hours. The sustained release of drug from the formulations of the invention enables a reduction in the frequency of administration, i.e. preferably a once-a-day administration.

The pain management drug, i.e. an opioid agonist, preferably oxycodone, formulations provided herein, when administered to a patient, e.g., a mammal, particularly a human patient, in a fasting state result in low peak to trough plasma concentration fluctuations and sufficient plasma concentrations to maintain a therapeutically effective analgesic effect over an extended period of time with reduced breakthrough pain so as to minimize the need for additional dosage administration within the extended time period, i.e. reduced rescue doses.

The process for making the pharmaceutical formulations of the present invention includes layering at least one layer of the desired pain management drug and optionally a suitable binding agent onto the surface of biologically inert pellets; i.e., non-pareil pellet (sugar and/or starch-based pellets). The drug layer may then optionally be substantially enveloped by a sealing coat layer. The drug layer or optional sealing coat layer is then substantially enveloped by an extended-release layer of water insoluble polymer, which is optionally coated with an enteric coating.

In preparing the formulations of the invention, the pain management drug layer may be sprayed onto non-pareil or other pellets that have been suspended in a fluidized bed, for example, or the pain management drug may be admixed with excipients to form drug-loaded granules. Preferably, the binder agent, such as hydroxypropylmethyl cellulose is dissolved in water to form a 5% to 30% (w/w) solution, preferably a 7% to 25% (w/w) solution and most preferably, an approximately 10% (w/w) solution, which is admixed with a solution of the drug(s) and any other desired pharmaceutical agent(s). The solution or suspension of drug(s) and binder agent is then applied onto the pellet using, for example, a fluid processor. Although some organic solvent may be used in the film coating application, the inclusion of organic solvents in the coating solutions used in the present methods is not required.

After the pellets are layered with pain management drug and binder agent to form drug loaded pellets, they may optionally be dried by air exposure, or other methods known in the art (although drying may occur spontaneously from air flow in the fluid bed processor). Similarly, any additional therapeutic agent (opioid antagonist or NSAID) is separately coated onto above-described inert pellets for addition to formulations comprising two or more components. Pellets obtained from the drug layering are then fluidized and sprayed with the water insoluble polymer coating to form the extended-release layer.

The water-insoluble polymer comprising the extended-release layer is generally prepared as a dispersion, optionally mixed with lubricant and/or talc and applied onto the prepared pellets. The total amount of water-insoluble polymer in the pellets is in the range of from about 2%-50% of the total weight of the prepared pellets, preferably about 4%-30% of the total weight of the prepared pellets, and most preferably about 5%-20% of the total weight of the prepared pellets. By varying the amount of extended-release controlling polymer on two or more oxycodone-layered pellets or oxycodone-loaded granules within this range, a desired sustained and controlled, i.e. extended, release of the therapeutic agent, e.g. oxycodone, is achieved by the oral dosage forms provided herein for up to 24 hours to provide pain relief for up to 24 hour.

At the final stage the pellets may be subjected to a curing process. The pellets are cured at a temperature in the range of from about 30° C. to about 50° C., preferably, from about 35° C. to about 45° C., and most preferably, about 40° C. for a period of about 5 to about 10 days and, preferably, about 7 days. Alternatively, pellets can be cured in a Fluid bed dryer at 50° C.-70° C. for 1-5 hours, most preferably at 60° C. for 2 hours.

The cured coated pellets preferably after addition of about 1%-2% dry talc to the coated pellets may be weighed out according to the total dose of pharmaceutical agent(s) to be administered to patients. Diluent may be added, such as, for example, dextrose, sorbitol, mannitol, microcrystalline cellulose, methocel ether, lactose, glyceryl palmitostearate, glyceryl stearate, glyceryl behenate, and combinations thereof, among other commonly used pharmaceutical diluents, and the mixture of coated pellets and diluents pressed into tablets. Alternatively, the mixture of the coated pellets alone can be encapsulated in a capsule, such as a hard gelatin capsule.

In another exemplary embodiment of the present invention, opioid layered pellets which are not coated with an extended-release layer may be added to formulations comprising at least one opioid layered pellet which is coated with an extended-release layer to form a two component system. Opioid layer coated pellets without an extended-release layer may be filled into a capsule solely, i.e. without the addition of an extended-release layer-coated opioid layered pellets. In further embodiments of the present invention, opioid layer coated pellets without an extended-release may be filled into a capsule together with one opioid layered pellet or a plurality of opioid layered pellets, each of which are coated with an extended-release layer, wherein each of the one opioid layered pellet or the plurality of opioid layered pellets (two or more pellets comprising an opioid layer coat and an extended-release layer) each have a different rate of release of the opioid agonist.

In additional embodiments of the formulations of the present invention, the dosage form (of at least one drug coated pellet or drug-loaded granule, at least one drug coated pellet or drug-loaded granule which is further coated with an extended-release layer, a combination thereof, or a plurality thereof) may further include a therapeutically effective amount of (a) at least one opioid antagonist, (b) at least one pain management drug, (c) at least one non-steroidal anti-inflammatory drug (NSAID), (d) at least one pain management drug and at least one NSAID, (e) at least one opioid antagonist and at least one pain management drug; (f) at least one opioid antagonist and at least one non-steroidal anti-inflammatory drug (NSAID); or (g) at least one opioid antagonist, at least one pain management drug and at least one non-steroidal anti-inflammatory drug (NSAID).

It is often desirable to add inert diluent when formulating the coated pellets into tablet form. The presence of pharmaceutical diluents, such as microcrystalline cellulose, lactose, methocel ether, glyceryl palmitostearate, glyceryl stearate, and/or glyceryl behemate, for example, in the pellet mixture serves to cushion the pellets so that they are not significantly ruptured during compression.

In general, the release rate of an opioid agonist from the pellets is dependent upon a number of factors including, inter alia, the overall structure and design of the layered pellet, the potency of the layered pellet, the type and amount of polymer admixed with the drug layer and type and amount of polymer and optional lubricant(s)/talc in the extended-release layer and the types of pellets. The pellets may be formulated into tablets or encapsulated in the desired dosage amount. Typical unit dosage amounts for the extended release opioid agonist formulations of the invention for oral administration include any dosage between about 10 and 400 mg, such as 15, 25, 30, 40, 50, 80, 100, 200, 300, 400 mg, depending on the specific opioid agonist of the formulation. The opioid agonist formulations of the invention are formulated to provide a pharmaceutically effective plasma concentration of drug(s) over an extended period of time after administration with low peak to trough plasma concentration fluctuations, i.e. a steady state blood plasma concentration profile. The extended release drug formulations of the present invention may be in a multiparticulate, e.g., pellet form, wherein an inert pellet is coated/layered with an opioid agonist, which may then further be coated with an extended release layer, which pellet(s) may then be placed into a capsule; or in a granule form, i.e. wherein the drug, e.g. opioid agonist, is mixed together with excipients and formulated into a dosage form, which granule may be coated with an extended release layer, to achieve the desired plasma concentrations of opioid agonist, preferably oxycodone.

The following examples are illustrative of the invention, and are not to be construed as limiting the invention.

### EXAMPLES

Examples 1-2: Oxycodone-Loaded Pellet Formulations

Amount in Grams

| Item # | Ingredients | Example 1 | Example 2 |
|---|---|---|---|
| 1 | Oxycodone Hydrochloride | 91.43 | 253.75 |
| 2 | Methocel E6 | 22.86 | 63.44 |
| 3 | Purified Water | 475.00 | 2042.5 |
| 4 | Sugar Spheres 25/30 Mesh | 685.71 | 432.81 |

| | | |
|---|---|---|
| Total* | 800.00* | 750.00* |

*Purified water is evaporated during the process and is not part of the final formulation.

Drug Layering Method
1. Methocel E6 10% solution is prepared in water by suspending Methocel E6 powder and mixing until the solution is achieved.
2. The active suspension is prepared by mixing Methocel E6 10% solution, oxycodone hydrochloride and purified water.
3. The active suspension is then applied onto sugar spheres using the fluid bed processor to produce oxycodone layered pellets.

Such layered pellets are also referred to herein as oxycodone-loaded pellets.

Examples 3-6: Extended-Release Oxycodone Pellet Formulations

| | | Amount in Grams | | | |
|---|---|---|---|---|---|
| Item # | Ingredients | Example 3 | Example 4 | Example 5 | Example 6 |
| 1 | Oxycodone Hydrochloride | 91.43 | 91.43 | 91.43 | 79.48 |
| 2 | Methocel E6 | 22.86 | 22.86 | 22.86 | 22.86 |
| 3 | Purified Water | 475.00 | 475.00 | 475.00 | 475.00 |
| 4 | Sugar Spheres 25/30 Mesh | 685.71 | 685.71 | 685.71 | 584.42 |
| 5 | Surelease ® E-7-19010 Solids | 40.00 | 60.00 | 80.00 | 68.18 |
| 6 | Purified Water | 226.67 | 340.00 | 453.00 | 386.35 |
| 7 | Methocel E6 | 0.00 | 0.00 | 0.00 | 11.62 |
| 8 | Purified Water | 0.00 | 0.00 | 0.00 | 220.78 |

| | | | | |
|---|---|---|---|---|
| Total* | 840.00* | 860.00* | 880.00* | 766.56* |

*Purified water is evaporated during the process and is not part of the final formulation.

Extended Release Layering Method
1. Methocel E6 10% solution is prepared in water by suspending Methocel E6 powder and mixing until the solution is achieved.
2. The active suspension is prepared by mixing Methocel E6 10% solution, oxycodone hydrochloride and purified water.
3. The active suspension is then applied onto sugar spheres using the fluid bed processor to achieve oxycodone layered pellets.
4. The coating suspension is prepared by mixing Surelease® and purified water is then applied onto the layered pellets to achieve the extended-release pellets. The pellets may optionally be subjected to a curing process.

5. In Example 6, a seal coating solution is prepared by mixing Methocel E6 powder into purified water and is then applied onto the extended-release pellets.

Examples 7-9: Extended-Release Oxycodone
Hydrochloride Pellet Formulations

| | | Amount in Grams | | |
|---|---|---|---|---|
| Item # | Ingredients | Example 7 | Example 8 | Example 9 |
| 1 | Oxycodone Hydrochloride | 97.14 | 97.14 | 97.14 |
| 2 | Methocel E6 | 24.29 | 24.29 | 24.29 |
| 3 | Purified Water | 475.00 | 475.00 | 475.00 |
| 4 | Sugar Spheres 25/30 Mesh | 728.57 | 728.57 | 728.57 |
| 5 | Eudragit NE30 D Solids | 42.56 | 63.75 | 90.56 |
| 6 | Calcium Stearate Powder | 12.77 | 19.13 | 27.17 |
| 7 | Simethicone Emulsion | 0.13 | 0.19 | 0.27 |
| | Solids | | | |
| 8 | Purified Water | 1.54 | 256.94 | 365.00 |

| | | | |
|---|---|---|---|
| Total* | 905.33* | 932.88* | 967.73* |

*Purified water is evaporated during the process and is not part of the final formulation.

Extended Release Layering Method
1. Methocel E6 10% solution is prepared in water by suspending Methocel E6 powder and mixing until the solution is achieved.
2. The active suspension is prepared by mixing Methocel E6 10% solution, oxycodone hydrochloride and purified water.
3. The active suspension is then applied onto sugar spheres using the Fluid bed processor to achieve oxycodone layered pellets.
4. The calcium stearate suspension is prepared by mixing simethicone emulsion, calcium stearate powder and purified water.
5. The coating suspension prepared by mixing calcium stearate suspension and Eudragit NE30D is then applied onto the layered pellets to achieve the extended-release pellets.

6. The extended-release pellets are then cured in the fluid bed processor for 2 hours at 60° C. or in an oven at 40° C. for 7 days.

Examples 10-12: Oxycodone Extended-
Release Pellet Formulations

| | | Amount in Grams | | |
|---|---|---|---|---|
| | | Example | Example | Example |
| Item # | Ingredients | 10 | 11 | 12 |
| 1 | Oxycodone Hydrochloride | 97.14 | 97.14 | 97.14 |
| 2 | Methocel E6 | 24.29 | 24.29 | 24.29 |
| 3 | Purified Water | 475.00 | 475.00 | 475.00 |
| 4 | Sugar Spheres 25/30 Mesh | 728.57 | 728.57 | 728.57 |
| 5 | Eudragit NE30 D Solids | 42.56 | 63.75 | 90.56 |
| 6 | Magnesium Stearate | 12.77 | 19.13 | 27.17 |
| | Powder | | | |
| 7 | Simethicone Emulsion | 0.13 | 0.19 | 0.27 |
| | Solids | | | |
| 8 | Purified Water | 171.54 | 256.94 | 365.00 |

| | | | |
|---|---|---|---|
| Total* | 905.33* | 932.88* | 967.73* |

*Purified water is evaporated during the process and is not part of the final formulation.

Extended Release Layering Method
1. Methocel E6 10% solution is prepared in water by suspending Methocel E6 powder and mixing until the solution is achieved.
2. The active suspension is prepared by mixing Methocel E6 10% solution, oxycodone hydrochloride and purified water.
3. The active suspension is then applied onto sugar spheres using the fluid bed processor to achieve oxycodone layered pellets.
4. The magnesium stearate suspension is prepared by mixing simethicone emulsion, magnesium stearate powder and purified water.
5. The coating suspension prepared by mixing magnesium stearate suspension and Eudragit NE30D is then applied onto the layered pellets to achieve the extended-release pellets.

6. The extended-release pellets are then cured in the fluid bed processor for 2 hours at 60° C. or in an oven at 40° C. for 7 days.

Examples 13-16: Oxycodone Extended-Release Pellet Formulations

| | | Amount in Grams | | | |
|---|---|---|---|---|---|
| | | Example | Example | Example | Example |
| Item # | Ingredient | 13 | 14 | 15 | 16 |
| 1 | Oxycodone Hydrochloride | 253.75 | 253.75 | 253.75 | 253.75 |
| 2 | Methocel E6 | 63.44 | 63.44 | 63.44 | 63.44 |
| 3 | Purified Water | 2042.50 | 2042.50 | 2042.50 | 2042.50 |
| 4 | Sugar Spheres 25/30 Mesh | 432.81 | 432.81 | 432.81 | 432.81 |
| 5 | Eudragit NE30 D Solids | 56.25 | 75.00 | 112.50 | 187.50 |
| 6 | Calcium Stearate Powder | 16.88 | 22.5 | 33.75 | 56.25 |
| 7 | Simethicone Emulsion Solids | 0.169 | 0.225 | 0.338 | 0.563 |
| 8 | Purified Water | 226.71 | 302.28 | 453.42 | 755.70 |

| | | | | |
|---|---|---|---|---|
| Total* | 823.29* | 847.73* | 896.59* | 994.31* |

*Purified water is evaporated during the process and is not part of the final formulation.

Extended Release Layering Method
1. Methocel E6 10% solution is prepared in water by suspending Methocel E6 powder and mixing until the solution is achieved.
2. The active suspension is prepared by mixing Methocel E6 10% solution, oxycodone hydrochloride and purified water.
3. The active suspension is then applied onto sugar spheres using the Fluid bed processor to achieve oxycodone layered pellets.
4. The calcium stearate suspension is prepared by mixing simethicone emulsion, calcium stearate powder and purified water.
5. The coating suspension prepared by mixing calcium stearate suspension and Eudragit NE30D is then applied onto the layered pellets to achieve the extended-release pellets.

6. The extended-release pellets are then cured in the fluid bed processor for 2 hours at 60° C. or in an oven at 40° C. for 7 days.

Examples 17-19: Oxycodone Extended-
Release Capsule Formulations. Strength: 40 mg
of Oxycodone Hydrochloride Per Capsule

| | | Amount in mg Per Capsule | | |
|---|---|---|---|---|
| | | Example | Example | Example |
| Item # | Ingredients | 17 | 18 | 19 |
| 1 | Example #1 Layered Pellets | 35.00 | 35.00 | 87.50 |
| 2 | Example #9 ER Pellets | 358.64 | 0 | 0 |
| 3 | Example #12 ER Pellets | 0 | 358.64 | 298.87 |

| | | | |
|---|---|---|---|
| Total | 393.64 | 393.64 | 386.37 |

Encapsulation Method

Fill the layered pellets and ER pellets into suitable size capsules.

Examples 20-21: Oxycodone Extended-
Release Capsule Formulations. Strength: 40 mg of
Oxycodone Hydrochloride Per Capsule

| | | Amount in mg | |
|---|---|---|---|
| | | Per Capsule | |
| Item # | Ingredient | Example 20 | Example 21 |
| 1 | Example #13 ER Pellets | 129.78 | 0 |
| 2 | Example #14 ER Pellets | 0 | 133.63 |

| | | |
|---|---|---|
| Total | 129.78 | 133.63 |

Encapsulation Method

Fill the ER pellets into suitable size capsules.

Examples 22-25: Oxycodone Extended-Release Capsule
Formulations.
Strength: 40 mg of Oxycodone Hydrochloride Per Capsule

| | | Amount in mg Per Capsule | | | |
|---|---|---|---|---|---|
| | | Example | Example | Example | Example |
| Item # | Ingredients | 22 | 23 | 24 | 25 |
| 1 | Example #13 | 12.98 | 12.98 | 64.89 | 0 |
| | ER Pellets | | | | |
| 2 | Example #14 | 120.27 | 0 | 0 | 66.82 |
| | ER Pellets | | | | |
| 3 | Example #15 | 0 | 127.20 | 70.67 | 70.67 |
| | ER Pellets | | | | |

| | | | | |
|---|---|---|---|---|
| Total | 133.25 | 140.18 | 135.56 | 137.49 |

Encapsulation Method

Fill the ER pellets into suitable size capsules.

Examples 26-32: Oxycodone Extended-Release Capsule Formulations.
Strength: 40 mg of Oxycodone Hydrochloride Per Capsule

| | | Amount in mg Per Capsule | | | | | | |
|---|---|---|---|---|---|---|---|---|
| | | Example | Example | Example | Example | Example | Example | Example |
| Item # | Ingredients | 26 | 27 | 28 | 29 | 30 | 31 | 32 |
| 1 | Example #2 | 17.72 | 11.81 | 11.81 | 11.81 | 17.72 | 0 | 0 |
| | Layered | | | | | | | |
| | Pellets | | | | | | | |
| 2 | Example #13 | 0 | 0 | 0 | 0 | 0 | 44.13 | 16.87 |
| | ER Pellets | | | | | | | |
| 3 | Example #14 | 0 | 0 | 60.13 | 13.36 | 0 | 0 | 42.76 |
| | ER Pellets | | | | | | | |
| 4 | Example #15 | 120.13 | 127.20 | 0 | 113.07 | 113.07 | 93.28 | 77.73 |
| | ER Pellets | | | | | | | |
| 5 | Example #16 | 0 | 0 | 70.53 | 0 | 7.84 | 0 | 0 |
| | ER Pellets | | | | | | | |

| | | | | | | | |
|---|---|---|---|---|---|---|---|
| Total | 137.85 | 139.01 | 142.47 | 138.24 | 138.63 | 137.41 | 137.36 |

Encapsulation Method

Fill the ER pellets into suitable size capsules.

### In Vitro Release and Dissolution of Oxycodone

Dissolution testing was performed on pellets prepared in Examples 2, 13-15, 17, 26, 29, and 31-32 (Tables 1 and 2). USP Apparatus I (Basket), 900 ml of deionized water, and 100 rpm were used as conditions for testing. Both oxycodone formulations of Examples 20 and 21, also tested for dissolution, contain 40 mg Oxycodone Hydrochloride per capsule. Table 3 provides the dissolution data of the formulation as per Example 20 in four different pH media using Apparatus I (basket) at 100 RPM. Table 4 provides the dissolution data as per Example 21 in four different pH media using Apparatus II (paddle) at 100 RPM. As seen from both the tables, the drug release from these formulations is pH independent. As can be seen in the tables, the present formulations demonstrate a sustained drug release for up to 24 hours.

TABLE 1
In Vitro Release and Dissolution Data of Oxycodone Hydrochloride
Pellets.
Method: USP Apparatus I (Basket); Deionized Water; 100 RPM;
900 ml; Pellets containing 40 mg of Oxycodone hydrochloride.

| | Percent Dissolved | | | | |
|---|---|---|---|---|---|
| Time | Example | Example | Example | Example | Example |
| (hours) | 2 | 13 | 14 | 15 | 17 |
| 1 | 100 | 2.8 | 3.0 | 0.6 | 0.3 |
| 2 | 100 | 11.1 | 8.7 | 0.8 | 0.6 |
| 4 | 100 | 39.0 | 24.8 | 8.2 | 1.9 |
| 8 | 100 | 89.2 | 64.8 | 29.6 | 12.2 |
| 12 | 100 | 100.3 | 85.3 | 55.5 | 27.8 |
| 16 | 100 | 102.8 | 96.3 | 75.1 | 46.0 |
| 24 | 100 | 104.8 | 103.8 | 95.4 | 74.4 |

TABLE 2
In Vitro Release and Dissolution Data of
Oxycodone Hydrochloride Pellets
Method: USP Apparatus I (Basket); Deionized Water;
100 RPM; 900 ml; Pellets Containing 40 mg
of Oxycodone hydrochloride.

| Time | Percent Dissolved | | | |
|---|---|---|---|---|
| (hours) | Example 26 | Example 29 | Example 31 | Example 32 |
| 1 | 19.0 | 11.2 | 0.5 | 3.1 |
| 2 | 19.6 | 13.2 | 4.8 | 6.8 |
| 4 | 27.6 | 21.4 | 21.4 | 19.7 |
| 8 | 49.5 | 46.0 | 53.2 | 51.2 |
| 12 | 74.0 | 71.5 | 73.5 | 72.2 |
| 16 | 90.6 | 88.3 | 90.2 | 89.9 |
| 24 | 99.3 | 100.5 | 104.4 | 103.4 |

TABLE 3
In Vitro Dissolution Profile of Oxycodone Hydrochloride Capsule
Formulation as per Example 20 at Different pHs.
Method: USP Apparatus I (Basket); 100 RPM; 900 ml of different
media; 40 mg of Oxycodone Hydrochloride per Capsule.

Percent Dissolved

| | | | Deionized | |
|---|---|---|---|---|
| Time (hours) | pH 1.2 | pH 4.5 | Water | pH 6.8 |
| 1 | 2.2 | 3.1 | 2.8 | 1.1 |
| 2 | 13.1 | 14.5 | 11.1 | 10.4 |
| 4 | 44.9 | 50.2 | 39 | 37.8 |
| 8 | 93.4 | 94.5 | 89.2 | 86.3 |
| 12 | 103.6 | 101.3 | 100.3 | 98.3 |
| 16 | 106.3 | 104.7 | 102.8 | 101.7 |
| 24 | 110 | 103.9 | 104.8 | 103.2 |

TABLE 4
In Vitro Dissolution Profile of Oxycodone Hydrochloride Capsule
Formulation as per Example 21 at Different pHs.
Method: USP Apparatus II (Paddle); 100 RPM; 900 ml of
different media; 40 mg of Oxycodone Hydrochloride per Capsule.

Percent Dissolved

| | | | Deionized | |
|---|---|---|---|---|
| Time (hour) | pH 1.2 | pH 4.5 | Water | pH 6.8 |
| 1 | 1.7 | 3.2 | 3 | 0.6 |
| 2 | 9 | 12 | 8.7 | 6.8 |
| 4 | 27 | 33.9 | 24.8 | 22.8 |
| 8 | 70.7 | 80.5 | 64.8 | 60.6 |
| 12 | 90 | 95.6 | 85.3 | 81.5 |
| 16 | 96.6 | 99 | 96.3 | 92.6 |
| 24 | 100.9 | 100.9 | 107.3 | 102.4 |

### Pharmacokinetic Study in Healthy Human Subjects Using Examples 20 and 21 Containing 40 mg of Oxycodone Hydrochloride Per Dose

A randomized four-way crossover pharmacokinetic study was conducted in 14 healthy non-tobacco using adult subjects in accordance with current FDA regulations. Thirteen of these subjects completed the study. The subjects included males and females of 18 years of age or older. Subjects were dosed after an overnight fast of at least ten hours. Subjects entered the clinic ten hours prior to dosing and were confined until at least 48 hours post-dose. Subjects were randomly assigned to the treatment groups according to a schedule generated by the study director. There was a seven day washout period between doses.

The following four products were tested in this study:
Treatment A: Example 20 from this invention containing 40 mg of Oxycodone Hydrochloride per capsule was administered one time with 240 ml of water. This product is intended to be a once-a-day product.
Treatment B: Example 21 from this invention containing 40 mg of Oxycodone Hydrochloride per capsule was administered one time with 240 ml of water. This product is intended to be a once-a-day product.
Treatment C: Oxycontin® Controlled Release Tablet containing 20 mg per tablet manufactured by Purdue Pharma L.P., Lot # DF81. A total of two tablets were administered, one at the initial time and second twelve hours after the first dose thus providing a total dose of 40 mg over a 24 hour period. The tablets were administered with 240 ml of water each time. The subjects were required to fast for at least two hours prior to the second dose. This product is currently marketed as a twice-a-day product.
Treatment D: Oxyfast® Oral Concentrated Solution containing 20 mg/ml of Oxycodone Hydrochloride manufactured by Purdue Pharma L.P., Lot # CC61. A single dose consisting of 1 ml of Oxyfast® containing 20 mg of Oxycodone Hydrochloride was added to 30 ml of water to prepare the dosing solution. After drinking this solution, subjects were required to drink an additional volume of 210 ml of water.

Other than the water used for dosing purposes, no fluids were allowed from one hour prior to and one hour after dosing. Standardized caffeine free meals or snacks were provided to all subjects during the confinement period at approximately 4, 9, 14, 25, 34, and 48 (optional release snack) hours after dosing.

Venous blood samples were collected before dosing and as a function of time for 48 hour period. For Treatments A, B and D, blood samples were collected as follows:
0.0 (pre-dose), 0.5, 1.0, 1.5, 2.0, 2.5, 3.0, 4.0, 5.0, 6.0, 8.0, 10.0 12.0, 14.0, 16.0, 20.0, 24.0, 36.0 and 48.0 hours post-dose (19 samples).
   For Treatment C, blood samples were collected as follows:
0.0 (pre-dose), 0.5, 1.0, 1.5, 2.0, 2.5, 3.0, 4.0, 5.0, 6.0, 8.0, 10.0 12.0, 12.5, 13.0, 14.0, 15.0, 16.0, 20.0, 24.0, 36.0 and 48.0 hours post-dose. (22 samples).

The samples were centrifuged at 4° C. at high speed (2500-2700 rpm) for 15 minutes. The resulting plasma was split into two halves and transferred to appropriate storage tubes and kept frozen at −20° C. until analyzed.

The following non-compartmental pharmacokinetic parameters were estimated from the plasma concentration profiles for each subject:
AUC₍ₒ₋ₜ₎ Area under the curve computed from the plasma concentration-time profiles from time zero to time of last quantifiable concentration.
AUC_{(0-Infinity)} Area under the curve computed from the plasma concentration-time profiles from time zero to infinity.
Cₘₐₓ Maximum plasma concentration observed over the entire sampling period.
Tₘₐₓ Time to attain Cₘₐₓ
Kₑₗ Apparent elimination constant
T_{1/2} Apparent elimination half life.

These parameters are summarized in Table 5 for the four test products evaluated in this study.

TABLE 5
Summary of Pharmacokinetic Data for Oxycodone
Hydrochloride Evaluated in Healthy Human Subjects.

| | Arithmetic Means (CV %) in 13 Subjects | | | |
|---|---|---|---|---|
| | Example 20; | Example 21; | Oxycontin ®; | Oxyfast ®; |
| PK | Treatment A | Treatment B | Treatment C | Treatment D |
| Parameter | n = 13 | n = 12 | n = 13 | n = 13 |
| AUC(0-inf) | 463.92 | 455.77 | 433.66 | 191.78 |
| (ng · hr/mL) | (24.05%) | (16.52%) | (21.06%) | (22.12%) |
| AUC(0-t) | 460.84 | 452.55 | 429.10 | 189.80 |
| (ng · hr/mL) | (24.03%) | (16.57%) | (20.86%) | (22.32%) |
| Cmax | 38.585 | 30.725 | 23.200 | 33.538 |
| (ng/mL) | (27.61%) | (23.58%) | (19.54%) | (44.15%) |
| T ½ | 5.88 | 5.92 | 5.53 | 6.41 |
| (hr) | (20.59%) | (12.12%) | (16.46%) | (45.93%) |
| Kel | 0.1226 | 0.1188 | 0.1284 | 0.1271 |
| (1/hr) | (20.37%) | (12.85%) | (15.62%) | (36.54%) |
| Tmax | 6.46 | 8.50 | 12.69 | 1.23 |
| (hr) | (17.44%) | (20.38%) | (42.72%) | (75.10%) |

Desired In-Vivo Profile & Necessary Percent Dissolved:

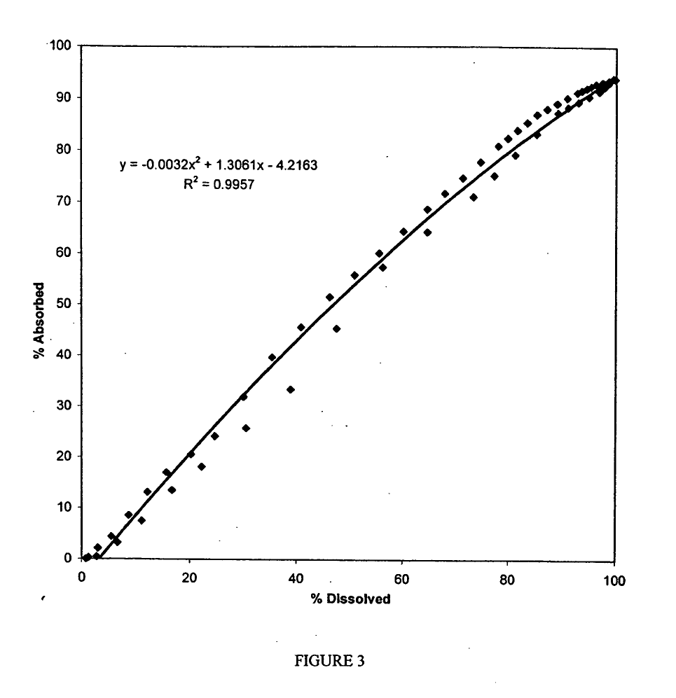

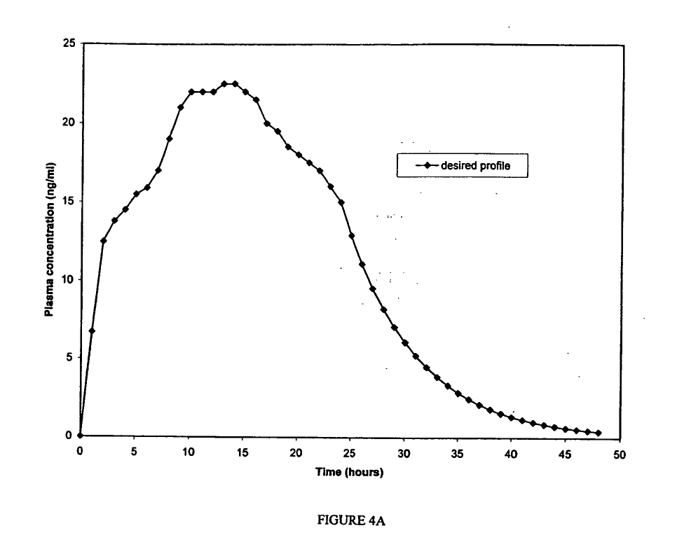

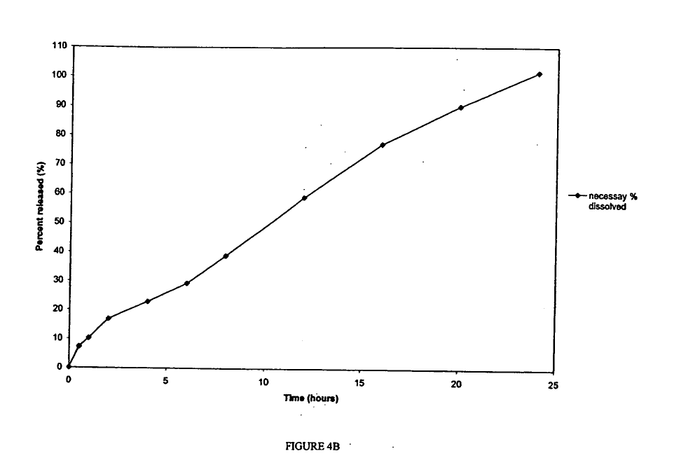

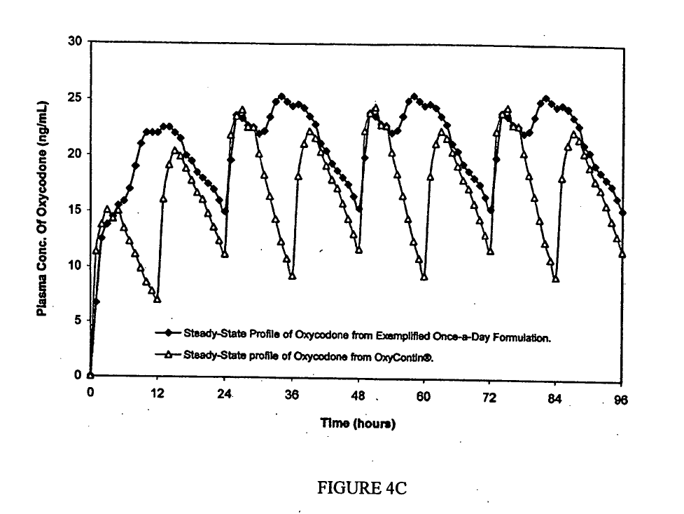

Based on the dissolution and pharmacokinetic data of Example 20 and Example 21, an IVIVC correlation was established. A one to one correlation was found as seen from FIG. 3. It was then possible to design a desired in-vivo profile of oxycodone which will have oxycodone concentration higher than the minimum effective concentration of oxycodone (between 5 to 10 ng/ml) at all time intervals especially between 20 to 24 hours after the dose administration. FIG. 4A shows a plot of mean plasma oxycodone concentration versus time under fasting condition representing simulated desired in-vivo profile. As seen from the figure, the oxycodone level does not fall below 10 ng/ml during at least a 24 hour time interval.

Again based on the IVIVC, it was possible to simulate the in-vitro dissolution profile for the desired in-vivo profile. FIG. 4B shows the necessary percentage dissolution profile in order to achieve the desired in-vivo profile.

Finally, steady-state simulation in-vivo profiles were generated (shown in FIG. 4C) for currently marketed product (OxyContin®) and for the once-a-day product. As seen from the figure that the oxycodone plasma concentration is higher than the minimum effective concentration throughout the profile. In addition, the peak to trough fluctuations from the once-a-day product are much lower than those from OxyContin®.

Pellets are prepared as described in any of Examples 3-16, in capsule form containing ER coated pellets, two different ER coated pellets or three different ER coated pellets, wherein any of the ER pellets may be combined with drug loaded pellets (as prepared in Examples 1 and 2), or in granule form, and formulated to contain a desired dosage of oxycodone hydrochloride from 10-400 mg. A capsule or granule is then orally administered to a patient in need of treatment of moderate to severe pain, e.g. in a patient afflicted with osteoarthritis or rheumatoid arthritis or any other disease in which such pain occurs. The dosage form can be administered upon need to the patient in either a fasting or fed state for relief of pain. This drug treatment may be continued as needed to treat and control the pain condition.

It should be understood that some modification, alteration and substitution is anticipated and expected from those skilled in the art without departing from the teachings of the invention. Accordingly, it is appropriate that the following claims be construed broadly and in a manner consistent with the scope and spirit of the invention.

1. An oral dosage form comprising:
at least one opioid agonist-loaded pellet or granule, wherein the opioid agonist comprises an opioid agonist or a pharmaceutically acceptable salt thereof, and
wherein the at least one opioid agonist-loaded pellet or granule is coated with an extended release layer, wherein the extended release layer comprises a first suitable amount of a water-insoluble polymer;
wherein the oral dosage form provides low peak to trough fluctuations of plasma concentration of the opioid agonist and a sufficient plasma concentration of the opioid agonist over an extended period of time to reduce incidence of breakthrough pain, and wherein the oral dosage form provides pain relief for up to 24 hours.

2. The oral dosage form of claim 1, wherein the opioid agonist comprises oxycodone or a pharmaceutically acceptable salt thereof.

3. The oral dosage form of claim 2, wherein the oxycodone or pharmaceutically acceptable salt thereof comprises between about 10 and 400 mgs of the oxycodone or pharmaceutically acceptable salt thereof.

4. The oral dosage form of claim 1, further comprising:
at least one second opioid agonist-loaded pellet or granule, wherein the opioid agonist comprises an opioid agonist or a pharmaceutically acceptable salt thereof, and
wherein the at least one second opioid agonist-loaded pellet or granule is coated with an extended release layer, wherein the extended release layer comprises a second suitable amount of a water-insoluble polymer, said second suitable amount of water-insoluble polymer being in an amount different than the first suitable amount of water-insoluble polymer.

5. The oral dosage form of claim 1, further comprising:
at least one opioid antagonist-loaded pellet or granule, wherein the opioid antagonist comprises an opioid antagonist or a pharmaceutically acceptable salt thereof.

6. The oral dosage form of claim 5, wherein the opioid antagonist is selected from the group consisting of naltrexone, naloxone, nalmephene, and nalorphine.

7. The oral dosage form of claim 1, further comprising:
at least one pain management drug-loaded pellet or granule or at least one non-steroidal anti-inflammatory drug (NSAID)-loaded pellet or granule.

8. The oral dosage form of claim 7, further comprising:
at least one opioid antagonist-loaded pellet or granule, wherein the opioid antagonist comprises an opioid antagonist or a pharmaceutically acceptable salt thereof.

9. The oral dosage form of claim 7, wherein the pain management drug is selected from the group consisting of oxycodone, codeine, morphine, hydromorphine, anilevidine, merperidine, methadone, levorphanol, pentazocine, propoxyphene, alfentanil, allyprodine, alphaprodine, anileridine, benzylmorphine, bezitramide, buprenorphine, butorphanol, clonitazene, cyclazocine, desomorphine, dextromoramide, dezocine, diampromide, dihydrocodeine, dihydromorphine, dimenoxadol, dimepheptanol, dimethylthiambutene, dioxaphetyl butyrate, dipipanone, eptazocine, ethoheptazine, ethylmethylthiambutene, ethylmorphine, etonitazene, fentanyl, heroin, hydrocodone, hydromorphone, hydroxypethidine, isomethadone, ketobemidone, levallorphan, levophenacylmorphan, lofentanil, meptazinol, metazocine, metopon, myrophine, nalbuphine, narceine, nicomorphine, norlevorphanol, normethadone, nalorphine, normorphine, norpipanone, opium, oxymorphone, papavretum, phenadoxone, phenomorphan, phenazocine, phenoperidine, piminodine, piritramide, propheptazine, promedol, properidine, propiram, sufentanil, tramadol, tilidine, salts thereof, and combinations thereof;
wherein the NSAID is selected from the group consisting of diclofenac, flufenamic acid, flurbiprofen, ibuprofen, indomethacin, ketoprofen, naproxen, phenylbutazone, sulindac, piroxicam, salicylic acid, acetylsalicylic acid, celecoxib, etodolac, fenoprofen, ketoralac, oxaprozin, nabumetone, tolmetin, and rofecoxib; and
wherein the opioid antagonist is selected from the group consisting of naltrexone, naloxone, nalmephene, and nalorphine.

10. The oral dosage form of claim 1, wherein the water-insoluble polymer is selected from the group consisting of alkylcellulose, an acrylic acid polymer, an acrylic acid copolymer, a methacrylic acid polymer, a methacrylic acid copolymer, shellac, zein, and hydrogenated vegetable oil.

11. The oral dosage form of claim 10, wherein the water-insoluble polymer comprises Eudragit NE 30D.

12. The oral dosage form of claim 1, wherein the extended release layer further comprises a lubricant.

13. The oral dosage form of claim 12, wherein the lubricant is selected from the group consisting of calcium stearate, magnesium stearate, zinc stearate, stearic acid, talc and a combination thereof.

14. The oral dosage form of claim 1, further comprising a sealing layer coated on the at least one opioid agonist-loaded pellet or granule.

15. The oral dosage form of claim 1, wherein the opioid-agonist layer further comprises a binder agent.

16. The oral dosage form of claim 15, wherein the binder agent is selected from the group consisting of hydroxypropylmethyl cellulose, hydroxyethyl cellulose, hydroxypropyl cellulose, methyl cellulose and polyvinyl pyrrolidone.

17. An oral dosage form comprising:
a first opioid agonist-loaded pellet or granule, wherein the opioid agonist comprises an opioid agonist or a pharmaceutically acceptable salt thereof, and wherein the first opioid agonist-loaded pellet or granule is coated with an extended release layer, wherein the extended release layer comprises a first suitable amount of a water-insoluble polymer; and
a second opioid agonist-loaded pellet or granule, wherein the opioid agonist comprises an opioid agonist or a pharmaceutically acceptable salt thereof, and
wherein the second opioid agonist-loaded pellet or granule is coated with an extended release layer, wherein the extended release layer comprises a second suitable amount of a water-insoluble polymer, said second suitable amount of water-insoluble polymer being different than the first suitable amount of water-insoluble polymer;
wherein the oral dosage form provides a dissolution rate having low peak to trough fluctuations of plasma concentration of the opioid agonist and a sufficient plasma concentration of the opioid agonist over an extended period of time to reduce incidence of breakthrough pain, and
wherein the oral dosage form provides pain relief for up to 24 hours.

18. The oral dosage form of claim 17, wherein the opioid agonist comprises oxycodone or a pharmaceutically acceptable salt thereof.

19. The oral dosage form of claim 18, wherein the oxycodone or pharmaceutically acceptable salt thereof comprises between about 10 and 400 mgs of the oxycodone or pharmaceutically acceptable salt thereof.

20. The oral dosage form of claim 17, further comprising:
at least one opioid antagonist-loaded pellet or granule, wherein the opioid antagonist comprises an opioid antagonist or a pharmaceutically acceptable salt thereof.

21. The oral dosage form of claim 20, wherein the opioid antagonist is selected from the group consisting of naltrexone, naloxone, nalmephene, and nalorphine.

22. The oral dosage form of claim 17, further comprising:
at least one pain management drug-loaded pellet or granule or at least one non-steroidal anti-inflammatory drug (NSAID)-loaded pellet or granule.

23. The oral dosage form of claim 22, further comprising:
at least one opioid antagonist-loaded pellet or granule, wherein the opioid antagonist comprises an opioid antagonist or a pharmaceutically acceptable salt thereof.

24. The oral dosage form of claim 23, wherein the pain management drug is selected from the group consisting of oxycodone, codeine, morphine, hydromorphine, anilevidine, merperidine, methadone, levorphanol, pentazocine, propoxyphene, alfentanil, allyprodine, alphaprodine, anileridine, benzylmorphine, bezitramide, buprenorphine, butorphanol, clonitazene, cyclazocine, desomorphine, dextromoramide, dezocine, diampromide, dihydrocodeine, dihydromorphine, dimenoxadol, dimepheptanol, dimethylthiambutene, dioxaphetyl butyrate, dipipanone, eptazocine, ethoheptazine, ethylmethylthiambutene, ethylmorphine, etonitazene, fentanyl, heroin, hydrocodone, hydromorphone, hydroxypethidine, isomethadone, ketobemidone, levallorphan, levophenacylmorphan, lofentanil, meptazinol, metazocine, metopon, myrophine, nalbuphine, narceine, nicomorphine, norlevorphanol, normethadone, nalorphine, normorphine, norpipanone, opium, oxymorphone, papavretum, phenadoxone, phenomorphan, phenazocine, phenoperidine, piminodine, piritramide, propheptazine, promedol, properidine, propiram, sufentanil, tramadol, tilidine, salts thereof, and combinations thereof;
wherein the NSAID is selected from the group consisting of diclofenac, flufenamic acid, flurbiprofen, ibuprofen, indomethacin, ketoprofen, naproxen, phenylbutazone, sulindac, piroxicam, salicylic acid, acetylsalicylic acid, celecoxib, etodolac, fenoprofen, ketoralac, oxaprozin, nabumetone, tolmetin, and rofecoxib; and
wherein the opioid antagonist is selected from the group consisting of naltrexone, naloxone, nalmephene, and nalorphine.

25. The oral dosage form of claim 17, wherein the water-insoluble polymer is selected from the group consisting of alkylcellulose, an acrylic acid polymer, an acrylic acid copolymer, a methacrylic acid polymer, a methacrylic acid copolymer, shellac, zein, and hydrogenated vegetable oil.

26. The oral dosage form of claim 25, wherein the water-insoluble polymer comprises Eudragit NE 30D.

27. The oral dosage form of claim 17, wherein the extended release layer further comprises a lubricant.

28. The oral dosage form of claim 27, wherein the lubricant is selected from the group consisting of calcium stearate, magnesium stearate, zinc stearate, stearic acid, talc and a combination thereof.

29. The oral dosage form of claim 17, further comprising a sealing layer coated on the first opioid agonist-loaded pellet or granule and on the second opioid agonist-loaded pellet or granule.

30. The oral dosage form of claim 17, wherein the opioid-agonist layer further comprises a binder agent.

31. The oral dosage form of claim 30, wherein the binder agent is selected from the group consisting of hydroxypropylmethyl cellulose, hydroxyethyl cellulose, hydroxypropyl cellulose, methyl cellulose and polyvinyl pyrrolidone.

32. An oral dosage form comprising:
a first opioid agonist-loaded pellet or granule, wherein the opioid agonist comprises an opioid agonist or a pharmaceutically acceptable salt thereof, and wherein the first opioid agonist-loaded pellet or granule is coated with an extended release layer, wherein the extended release layer comprises a first suitable amount of a water-insoluble polymer; and
a second opioid agonist-loaded pellet or granule, wherein the opioid agonist comprises an opioid agonist or a pharmaceutically acceptable salt thereof, wherein the second opioid agonist-loaded pellet or granule is coated with an extended release layer, wherein the extended release layer comprises a second suitable amount of a water-insoluble polymer, said second suitable amount of water-insoluble polymer being different than the first suitable amount of water-insoluble polymer; and
a third opioid agonist-loaded pellet or granule, wherein the opioid agonist comprises an opioid agonist or a pharmaceutically acceptable salt thereof, wherein the third opioid agonist-loaded pellet or granule is coated with an extended release layer, wherein the extended release layer comprises a third suitable amount of a water-insoluble polymer, said third suitable amount of water-insoluble polymer being different than the first suitable amount of water-insoluble polymer and different than the second amount of water-insoluble polymer;
wherein the oral dosage form provides low peak to trough fluctuations of plasma concentration of the opioid agonist and a sufficient plasma concentration of the opioid agonist over an extended period of time to reduce incidence of breakthrough pain, and
wherein the oral dosage form provides pain relief for up to 24 hours.

33. The oral dosage form of claim 32, wherein the opioid agonist comprises oxycodone or a pharmaceutically acceptable salt thereof.

34. The oral dosage form of claim 33, wherein the oxycodone or pharmaceutically acceptable salt thereof comprises between about 10 and 400 mgs of the oxycodone or pharmaceutically acceptable salt thereof.

35. The oral dosage form of claim 32, further comprising:
at least one opioid antagonist-loaded pellet or granule, wherein the opioid antagonist comprises an opioid antagonist or a pharmaceutically acceptable salt thereof.

36. The oral dosage form of claim 35, wherein the opioid antagonist is selected from the group consisting of naltrexone, naloxone, and nalmephene.

37. The oral dosage form of claim 32, further comprising:
at least one pain management drug-loaded pellet or granule or at least one non-steroidal anti-inflammatory drug (NSAID)-loaded pellet or granule.

38. The oral dosage form of claim 37, further comprising:
at least one opioid antagonist-loaded pellet or granule, wherein the opioid antagonist comprises an opioid antagonist or a pharmaceutically acceptable salt thereof.

39. The oral dosage form of claim 38, wherein the pain management drug is selected from the group consisting of oxycodone, codeine, morphine, hydromorphine, anilevidine, merperidine, methadone, levorphanol, pentazocine, propoxyphene, alfentanil, allyprodine, alphaprodine, anileridine, benzylmorphine, bezitramide, buprenorphine, butorphanol, clonitazene, cyclazocine, desomorphine, dextromoramide, dezocine, diampromide, dihydrocodeine, dihydromorphine, dimenoxadol, dimepheptanol, dimethylthiambutene, dioxaphetyl butyrate, dipipanone, eptazocine, ethoheptazine, ethylmethylthiambutene, ethylmorphine, etonitazene, fentanyl, heroin, hydrocodone, hydromorphone, hydroxypethidine, isomethadone, ketobemidone, levallorphan, levophenacylmorphan, lofentanil, meptazinol, metazocine, metopon, myrophine, nalbuphine, narceine, nicomorphine, norlevorphanol, normethadone, nalorphine, normorphine, norpipanone, opium, oxymorphone, papavretum, phenadoxone, phenomorphan, phenazocine, phenoperidine, piminodine, piritramide, propheptazine, promedol, properidine, propiram, sufentanil, tramadol, tilidine, salts thereof, and combinations thereof;
wherein the NSAID is selected from the group consisting of diclofenac, flufenamic acid, flurbiprofen, ibuprofen, indomethacin, ketoprofen, naproxen, phenylbutazone, sulindac, piroxicam, salicylic acid, acetylsalicylic acid, celecoxib, etodolac, fenoprofen, ketoralac, oxaprozin, nabumetone, tolmetin, and rofecoxib; and
wherein the opioid antagonist is selected from the group consisting of naltrexone, naloxone, nalmephene and nalorphine.

40. The oral dosage form of claim 32, wherein the water-insoluble polymer is selected from the group consisting of alkylcellulose, an acrylic acid polymer, an acrylic acid copolymer, a methacrylic acid polymer, a methacrylic acid copolymer, shellac, zein, and hydrogenated vegetable oil.

41. The oral dosage form of claim 40, wherein the water-insoluble polymer comprises Eudragit NE 30D.

42. The oral dosage form of claim 32, wherein the extended release layer further comprises a lubricant.

43. The oral dosage form of claim 42, wherein the lubricant is selected from the group consisting of calcium stearate, magnesium stearate, zinc stearate, stearic acid, talc and a combination thereof.

44. The oral dosage form of claim 32, further comprising a sealing layer coated on the first opioid agonist-loaded pellet or granule, on the second opioid agonist-loaded pellet or granule, and on the third opioid agonist-loaded pellet or granule.

45. The oral dosage form of claim 32, wherein the opioid-agonist layer further comprises a binder agent.

46. The oral dosage form of claim 45, wherein the binder agent is selected from the group consisting of hydroxypropylmethyl cellulose, hydroxyethyl cellulose, hydroxypropyl cellulose, methyl cellulose and polyvinyl pyrrolidone.

47. An oral dosage form including:
a first opioid agonist-loaded pellet or granule, wherein the opioid agonist comprises an opioid agonist or a pharmaceutically acceptable salt thereof; and
a second opioid agonist-loaded pellet or granule, wherein the opioid agonist comprises an opioid agonist or a pharmaceutically acceptable salt thereof, and
wherein the second opioid agonist-loaded pellet or granule is coated with an extended release layer, wherein the extended release layer comprises a first suitable amount of a water-insoluble polymer;
wherein the oral dosage form provides low peak to trough fluctuations of plasma concentration of the opioid agonist and a sufficient plasma concentration of the opioid agonist over an extended period of time to reduce incidence of breakthrough pain, and
wherein the oral dosage form provides pain relief for up to 24 hours.

48. The oral dosage form of claim 47, wherein the opioid agonist comprises oxycodone or a pharmaceutically acceptable salt thereof.

49. The oral dosage form of claim 48, wherein the oxycodone or pharmaceutically acceptable salt thereof comprises between about 10 and 400 mgs of the oxycodone or pharmaceutically acceptable salt thereof.

50. The oral dosage form of claim 47, further comprising:
a third opioid agonist-loaded pellet or granule, wherein the opioid agonist comprises an opioid agonist or a pharmaceutically acceptable salt thereof, and
wherein the third opioid agonist-loaded pellet or granule is coated with an extended release layer, wherein the extended release layer comprises a second suitable amount of a water-insoluble polymer, said water-insoluble polymer being in an amount different than the first water-insoluble polymer.

51. The oral dosage form of claim 47, further comprising:
at least one opioid antagonist-loaded pellet or granule, wherein the opioid antagonist comprises an opioid antagonist or a pharmaceutically acceptable salt thereof.

52. The oral form of claim 51, wherein the opioid antagonist is selected from the group consisting of naltrexone, naloxone, nalmephene, and nalorphine.

53. The oral dosage form of claim 47, further comprising:
at least one pain management drug-loaded pellet or granule or at least one non-steroidal anti-inflammatory drug (NSAID)-loaded pellet or granule.

54. The oral dosage form of claim 53, further comprising:
at least one opioid antagonist-loaded pellet or granule, wherein the opioid antagonist comprises an opioid antagonist or a pharmaceutically acceptable salt thereof.

55. The oral dosage form of claim 54, wherein the pain management drug is selected from the group consisting of oxycodone, codeine, morphine, hydromorphine, anilevidine, merperidine, methadone, levorphanol, pentazocine, propoxyphene, alfentanil, allyprodine, alphaprodine, anileridine, benzylmorphine, bezitramide, buprenorphine, butorphanol, clonitazene, cyclazocine, desomorphine, dextromoramide, dezocine, diampromide, dihydrocodeine, dihydromorphine, dimenoxadol, dimepheptanol, dimethylthiambutene, dioxaphetyl butyrate, dipipanone, eptazocine, ethoheptazine, ethylmethylthiambutene, ethylmorphine, etonitazene, fentanyl, heroin, hydrocodone, hydromorphone, hydroxypethidine, isomethadone, ketobemidone, levallorphan, levophenacylmorphan, lofentanil, meptazinol, metazocine, metopon, myrophine, nalbuphine, narceine, nicomorphine, norlevorphanol, normethadone, nalorphine, normorphine, norpipanone, opium, oxymorphone, papavretum, phenadoxone, phenomorphan, phenazocine, phenoperidine, piminodine, piritramide, propheptazine, promedol, properidine, propiram, sufentanil, tramadol, tilidine, salts thereof, and combinations thereof;
wherein the NSAID is selected from the group consisting of diclofenac, flufenamic acid, flurbiprofen, ibuprofen, indomethacin, ketoprofen, naproxen, phenylbutazone, sulindac, piroxicam, salicylic acid, acetylsalicylic acid, celecoxib, etodolac, fenoprofen, ketoralac, oxaprozin, nabumetone, tolmetin, and rofecoxib; and
wherein the opioid antagonist is selected from the group consisting of naltrexone, naloxone, nalmephene, and nalorphine.

56. The oral dosage form of claim 47, wherein the water-insoluble polymer is selected from the group consisting of alkylcellulose, an acrylic acid polymer, an acrylic acid copolymer, a methacrylic acid polymer, a methacrylic acid copolymer, shellac, zein, and hydrogenated vegetable oil.

57. The oral dosage form of claim 56, wherein the water-insoluble polymer comprises Eudragit NE 30D.

58. The oral dosage form of claim 47, wherein the extended release layer further comprises a lubricant.

59. The oral dosage form of claim 58, wherein the lubricant is selected from the group consisting of calcium stearate, magnesium stearate, zinc stearate, stearic acid, talc and a combination thereof.

60. The oral dosage form of claim 47, further comprising a sealing layer coated on the first opioid agonist-loaded pellet or granule and on the second opioid agonist-loaded pellet or granule.

61. The oral dosage form of claim 47, wherein the opioid-agonist layer further comprises a binder agent.

62. The oral dosage form of claim 61, wherein the binder agent is selected from the group consisting of hydroxypropylmethyl cellulose, hydroxyethyl cellulose, hydroxypropyl cellulose, methyl cellulose and polyvinyl pyrrolidone.

63. An oral dosage form including:
a first opioid agonist-loaded pellet or granule, wherein the opioid agonist comprises an opioid agonist or a pharmaceutically acceptable salt thereof; and
a second opioid agonist-loaded pellet or granule, wherein the opioid agonist comprises an opioid agonist or a pharmaceutically acceptable salt thereof, wherein the second opioid agonist-loaded pellet or granule is coated with an extended release layer, wherein the extended release layer comprises a first suitable amount of a water-insoluble polymer; and
a third opioid agonist-loaded pellet or granule, wherein the opioid agonist comprises an opioid agonist or a pharmaceutically acceptable salt thereof, and wherein the third opioid agonist-loaded pellet or granule is coated with an extended release layer, wherein the extended release layer comprises a second suitable amount of a water-insoluble polymer, said second suitable amount of water-insoluble polymer being different than the first suitable amount of water-insoluble polymer;
wherein the oral dosage form provides low peak to trough fluctuations of plasma concentration of the opioid agonist and a sufficient plasma concentration of the opioid agonist over an extended period of time to reduce incidence of breakthrough pain, and
wherein the oral dosage form provides pain relief for up to 24 hours.

64. The oral dosage form of claim 63, wherein the opioid agonist comprises oxycodone or a pharmaceutically acceptable salt thereof.

65. The oral dosage form of claim 64, wherein the oxycodone or pharmaceutically acceptable salt thereof comprises between about 10 and 400 mgs of the oxycodone or pharmaceutically acceptable salt thereof.

66. The oral dosage form of claim 63, further comprising:
at least one opioid antagonist-loaded pellet or granule, wherein the opioid antagonist comprises an opioid antagonist or a pharmaceutically acceptable salt thereof.

67. The oral form of claim 66, wherein the opioid antagonist is selected from the group consisting of naltrexone, naloxone, nalmephene, and nalorphine.

68. The oral dosage form of claim 63, further comprising:
at least one pain management drug-loaded pellet or granule or at least one non-steroidal anti-inflammatory drug (NSAID)-loaded pellet or granule.

69. The oral dosage form of claim 68, further comprising:
at least one opioid antagonist-loaded pellet or granule, wherein the opioid antagonist comprises an opioid antagonist or a pharmaceutically acceptable salt thereof.

70. The oral dosage form of claim 69, wherein the pain management drug is selected from the group consisting of oxycodone, codeine, morphine, hydromorphine, anilevidine, merperidine, methadone, levorphanol, pentazocine, propoxyphene, alfentanil, allyprodine, alphaprodine, anileridine, benzylmorphine, bezitramide, buprenorphine, butorphanol, clonitazene, cyclazocine, desomorphine, dextromoramide, dezocine, diampromide, dihydrocodeine, dihydromorphine, dimenoxadol, dimepheptanol, dimethylthiambutene, dioxaphetyl butyrate, dipipanone, eptazocine, ethoheptazine, ethylmethylthiambutene, ethylmorphine, etonitazene, fentanyl, heroin, hydrocodone, hydromorphone, hydroxypethidine, isomethadone, ketobemidone, levallorphan, levophenacylmorphan, lofentanil, meptazinol, metazocine, metopon, myrophine, nalbuphine, narceine, nicomorphine, norlevorphanol, normethadone, nalorphine, normorphine, norpipanone, opium, oxymorphone, papavretum, phenadoxone, phenomorphan, phenazocine, phenoperidine, piminodine, piritramide, propheptazine, promedol, properidine, propiram, sufentanil, tramadol, tilidine, salts thereof, and combinations thereof;
wherein the NSAID is selected from the group consisting of diclofenac, flufenamic acid, flurbiprofen, ibuprofen, indomethacin, ketoprofen, naproxen, phenylbutazone, sulindac, piroxicam, salicylic acid, acetylsalicylic acid, celecoxib, etodolac, fenoprofen, ketoralac, oxaprozin, nabumetone, tolmetin, and rofecoxib; and
wherein the opioid antagonist is selected from the group consisting of naltrexone, naloxone, nalmephene, and nalorphine.

71. The oral dosage form of claim 63, wherein the water-insoluble polymer is selected from the group consisting of alkylcellulose, an acrylic acid polymer, an acrylic acid copolymer, a methacrylic acid polymer, a methacrylic acid copolymer, shellac, zein, and hydrogenated vegetable oil.

72. The oral dosage form of claim 71, wherein the water-insoluble polymer comprises Eudragit NE 30D.

73. The oral dosage form of claim 63, wherein the extended release layer further comprises a lubricant.

74. The oral dosage form of claim 73, wherein the lubricant is selected from the group consisting of calcium stearate, magnesium stearate, zinc stearate, stearic acid, talc and a combination thereof.

75. The oral dosage form of claim 63, further comprising a sealing layer coated on the opioid agonist layer of the first opioid agonist-loaded pellet or granule, on the second opioid agonist-loaded pellet or granule, and on the third opioid agonist-loaded pellet or granule.

76. The oral dosage form of claim 63, wherein the opioid-agonist layer further comprises a binder agent.

77. The oral dosage form of claim 76, wherein the binder agent is selected from the group consisting of hydroxypropylmethyl cellulose, hydroxyethyl cellulose, hydroxypropyl cellulose, methyl cellulose and polyvinyl pyrrolidone.

78. An oral dosage form including:
a first opioid agonist-loaded pellet or granule, wherein the opioid agonist comprises an opioid agonist or a pharmaceutically acceptable salt thereof; and
a second opioid agonist-loaded pellet or granule, wherein the opioid agonist comprises an opioid agonist or a pharmaceutically acceptable salt thereof, wherein the second opioid agonist-loaded pellet or granule is coated with an extended release layer, wherein the extended release layer comprises a first suitable amount of a water-insoluble polymer;
a third opioid agonist-loaded pellet or granule, wherein the opioid agonist comprises an opioid agonist or a pharmaceutically acceptable salt thereof, and wherein the third opioid agonist-loaded pellet or granule is coated with an extended release layer, wherein the extended release layer comprises a second suitable amount of a water-insoluble polymer, said second suitable amount of water-insoluble polymer being different than the first suitable amount of water-insoluble polymer; and
a fourth opioid agonist-loaded pellet or granule, wherein the opioid agonist comprises an opioid agonist or a pharmaceutically acceptable salt thereof, and wherein the fourth opioid agonist-loaded pellet or granule is coated with an extended release layer, wherein the extended release layer comprises a third suitable amount of a water-insoluble polymer, said third suitable amount of water-insoluble polymer being different than the first suitable amount of water-insoluble polymer and being different than the second suitable amount of water-insoluble polymer;
wherein the oral dosage form provides low peak to trough fluctuations of plasma concentration of the opioid agonist and a sufficient plasma concentration of the opioid agonist over an extended period of time to reduce incidence of breakthrough pain, and
wherein the oral dosage form provides pain relief for up to 24 hours.

79. The oral dosage form of claim 78, wherein the opioid agonist comprises oxycodone or a pharmaceutically acceptable salt thereof.

80. The oral dosage form of claim 79, wherein the oxycodone or pharmaceutically acceptable salt thereof comprises between about 10 and 400 mgs of the oxycodone or pharmaceutically acceptable salt thereof.

81. The oral dosage form of claim 78, further comprising:
at least one opioid antagonist-loaded pellet or granule, wherein the opioid antagonist comprises an opioid antagonist or a pharmaceutically acceptable salt thereof.

82. The oral form of claim 81, wherein the opioid antagonist is selected from the group consisting of naltrexone, naloxone, nalmephene, and nalorphine.

83. The oral dosage form of claim 78, further comprising:
at least one pain management drug-loaded pellet or granule or at least one non-steroidal anti-inflammatory drug (NSAID)-loaded pellet or granule.

84. The oral dosage form of claim 83, further comprising:
at least one opioid antagonist-loaded pellet or granule, wherein the opioid antagonist comprises an opioid antagonist or a pharmaceutically acceptable salt thereof.

85. The oral dosage form of claim 84, wherein the pain management drug is selected from the group consisting of oxycodone, codeine, morphine, hydromorphine, anilevidine, merperidine, methadone, levorphanol, pentazocine, propoxyphene, alfentanil, allyprodine, alphaprodine, anileridine, benzylmorphine, bezitramide, buprenorphine, butorphanol, clonitazene, cyclazocine, desomorphine, dextromoramide, dezocine, diampromide, dihydrocodeine, dihydromorphine, dimenoxadol, dimepheptanol, dimethylthiambutene, dioxaphetyl butyrate, dipipanone, eptazocine, ethoheptazine, ethylmethylthiambutene, ethylmorphine, etonitazene, fentanyl, heroin, hydrocodone, hydromorphone, hydroxypethidine, isomethadone, ketobemidone, levallorphan, levophenacylmorphan, lofentanil, meptazinol, metazocine, metopon, myrophine, nalbuphine, narceine, nicomorphine, norlevorphanol, normethadone, nalorphine, normorphine, norpipanone, opium, oxymorphone, papavretum, phenadoxone, phenomorphan, phenazocine, phenoperidine, piminodine, piritramide, propheptazine, promedol, properidine, propiram, sufentanil, tramadol, tilidine, salts thereof, and combinations thereof;
wherein the NSAID is selected from the group consisting of diclofenac, flufenamic acid, flurbiprofen, ibuprofen, indomethacin, ketoprofen, naproxen, phenylbutazone, sulindac, piroxicam, salicylic acid, acetylsalicylic acid, celecoxib, etodolac, fenoprofen, ketoralac, oxaprozin, nabumetone, tolmetin, and rofecoxib; and
wherein the opioid antagonist is selected from the group consisting of naltrexone, naloxone, nalmephene, and nalorphine.

86. The oral dosage form of claim 78, wherein the water-insoluble polymer is selected from the group consisting of alkylcellulose, an acrylic acid polymer, an acrylic acid copolymer, a methacrylic acid polymer, a methacrylic acid copolymer, shellac, zein, and hydrogenated vegetable oil.

87. The oral dosage form of claim 86, wherein the water-insoluble polymer comprises Eudragit NE 30D.

88. The oral dosage form of claim 78, wherein the extended release layer further comprises a lubricant.

89. The oral dosage form of claim 88, wherein the lubricant is selected from the group consisting of calcium stearate, magnesium stearate, zinc stearate, stearic acid, talc and a combination thereof.

90. The oral dosage form of claim 78, further comprising a sealing layer coated on the first opioid agonist-loaded pellet or granule, on the second opioid agonist-loaded pellet or granule, and on the third opioid agonist-loaded pellet or granule.

91. The oral dosage form of claim 78, wherein the opioid-agonist layer further comprises a binder agent.

92. The oral dosage form of claim 91, wherein the binder agent is selected from the group consisting of hydroxypropylmethyl cellulose, hydroxyethyl cellulose, hydroxypropyl cellulose, methyl cellulose and polyvinyl pyrrolidone.

93. A method of preparing an extended release oral dosage form of an opioid agonist, said method comprising:
coating at least one biologically inert pellet with a dose of the opioid agonist or a pharmaceutically acceptable salt thereof to form the opioid agonist-loaded pellet or admixing a dose of an opioid agonist with inert excipients to form an opioid agonist-loaded granule; and
coating the opioid agonist-loaded pellet or granule with an extended release layer, wherein the extended release layer comprises a first suitable amount of a water-insoluble polymer.

94. The oral dosage form of claim 93, wherein the opioid agonist comprises oxycodone or a pharmaceutically acceptable salt thereof.

95. The method of claim 94, wherein oxycodone or salt thereof comprises between about 10 and 400 mgs of the oxycodone or pharmaceutically acceptable salt thereof.

96. The method of claim 93, further comprising adding to the extended release oral dosage form of the opioid agonist an opioid antagonist loaded pellet or granule, wherein the opioid antagonist layer comprises an opioid antagonist or a pharmaceutically acceptable salt.

97. The method of claim 96, wherein the opioid antagonist is selected from the group consisting of naltrexone, naloxone, nalmephene, and nalorphine.

98. The method of claim 93, further comprising adding to the extended release oral dosage form of the opioid agonist a drug loaded pellet or granule, wherein the drug loaded pellet or granule is loaded with at least one pain management drug or at least one non-steroidal anti-inflammatory drug (NSAID).

99. The method of claim 93, further comprising adding to the extended release oral dosage form of the opioid agonist a first drug loaded pellet or granule and a second a drug loaded pellet or granule, wherein the first drug loaded pellet or granule is loaded with at least one pain management drug or at least one non-steroidal anti-inflammatory drug (NSAID), and the second drug loaded pellet or granule is loaded with at least one opioid antagonist.

100. The method of claim 99, wherein the pain management drug is selected from the group consisting of oxycodone, codeine, morphine, hydromorphine, anilevidine, merperidine, methadone, levorphanol, pentazocine, propoxyphene, alfentanil, allyprodine, alphaprodine, anileridine, benzylmorphine, bezitramide, buprenorphine, butorphanol, clonitazene, cyclazocine, desomorphine, dextromoramide, dezocine, diampromide, dihydrocodeine, dihydromorphine, dimenoxadol, dimepheptanol, dimethylthiambutene, dioxaphetyl butyrate, dipipanone, eptazocine, ethoheptazine, ethylmethylthiambutene, ethylmorphine, etonitazene, fentanyl, heroin, hydrocodone, hydromorphone, hydroxypethidine, isomethadone, ketobemidone, levallorphan, levophenacylmorphan, lofentanil, meptazinol, metazocine, metopon, myrophine, nalbuphine, narceine, nicomorphine, norlevorphanol, normethadone, nalorphine, normorphine, norpipanone, opium, oxymorphone, papavretum, phenadoxone, phenomorphan, phenazocine, phenoperidine, piminodine, piritramide, propheptazine, promedol, properidine, propiram, sufentanil, tramadol, tilidine, salts thereof, and combinations thereof;
wherein the NSAID is selected from the group consisting of diclofenac, flufenamic acid, flurbiprofen, ibuprofen, indomethacin, ketoprofen, naproxen, phenylbutazone, sulindac, piroxicam, salicylic acid, acetylsalicylic acid, celecoxib, etodolac, fenoprofen, ketoralac, oxaprozin, nabumetone, tolmetin, and rofecoxib; and
wherein the opioid antagonist is selected from the group consisting of naltrexone, naloxone, nalmephene, and nalorphine.

101. The method of claim 93, wherein the water-insoluble polymer is selected from the group consisting of alkylcellulose, an acrylic acid polymer, an acrylic acid copolymer, a methacrylic acid polymer, a methacrylic acid copolymer, shellac, zein, and hydrogenated vegetable oil.

102. The method of claim 101, wherein the water-insoluble polymer comprises Eudragit NE 30D.

103. The method of claim 93, wherein the extended release layer further comprises a lubricant.

104. The method of claim 103, wherein the lubricant is selected from the group consisting of calcium stearate, magnesium stearate, zinc stearate, stearic acid, talc and a combination thereof.

105. The method of claim 59, further comprising coating the opioid agonist loaded pellet or granule with a sealing layer.

106. The method of claim 105, wherein the opioid-agonist layer further comprises a binder agent.

107. The oral dosage form of claim 106, wherein the binder agent is selected from the group consisting of hydroxypropylmethyl cellulose, hydroxyethyl cellulose, hydroxypropyl cellulose, methyl cellulose and polyvinyl pyrrolidone.

108. The method of claim 93, wherein the oral dosage form is in a capsule or a granule form.

109. The method of claim 93, further comprising adding at least one second opioid agonist loaded pellet or granule to the oral dosage form, wherein the at least one second opioid agonist comprises an opioid agonist or a pharmaceutically acceptable salt thereof, said at least one second opioid agonist loaded pellet or granule comprising an extended release layer, wherein the extended release layer comprises a second suitable amount of a water-insoluble polymer, said second suitable amount of water-insoluble polymer being different than the first suitable amount of water-insoluble polymer.

110. The method of claim 109, wherein the opioid agonist comprises oxycodone or a pharmaceutically acceptable salt thereof.

111. The oral dosage form of claim 110, wherein the oxycodone or pharmaceutically acceptable salt thereof comprises between about 10 and 400 mgs of the oxycodone or pharmaceutically acceptable salt thereof.

112. The method of claim 93, further comprising adding at least one third opioid agonist loaded pellet or granule to the oral dosage form, wherein the at least one third opioid agonist comprises an opioid agonist or a pharmaceutically acceptable salt thereof, said at least one third opioid agonist loaded pellet or granule comprising an extended release layer, wherein the extended release layer comprises a third suitable amount of a water-insoluble polymer, said third suitable amount of water-insoluble polymer being different than the first suitable amount of water-insoluble polymer and being different than the second suitable amount of water-insoluble polymer.

113. The method of claim 112, wherein the opioid agonist comprises oxycodone or a pharmaceutically acceptable salt thereof.

114. The oral dosage form of claim 113, wherein the oxycodone or pharmaceutically acceptable salt thereof comprises between about 10 and 400 mgs of the oxycodone or pharmaceutically acceptable salt thereof.

115. The method of claim 93, further comprising adding at least one second opioid agonist-loaded pellet or granule to the oral dosage, wherein the at least one second opioid agonist comprises an opioid agonist or a pharmaceutically acceptable salt thereof.

116. The method of claim 115, wherein the opioid agonist comprises oxycodone or a pharmaceutically acceptable salt thereof.

117. The oral dosage form of claim 116, wherein the oxycodone or pharmaceutically acceptable salt thereof comprises between about 10 and 400 mgs of the oxycodone or pharmaceutically acceptable salt thereof.

118. A method of treating pain in a subject in need thereof, said method comprising orally administering to the subject an oral dosage form of at least one opioid agonist-loaded pellet or granule, wherein the opioid agonist comprises an opioid agonist or a pharmaceutically acceptable salt thereof, and
wherein the at least one opioid agonist-loaded pellet or granule is coated with an extended release layer, wherein the extended release layer comprises a first suitable amount of a water-insoluble polymer;
wherein the oral dosage form provides low peak to trough fluctuations of plasma concentration of the opioid agonist and a sufficient plasma concentration of the opioid agonist over an extended period of time to reduce incidence of breakthrough pain, and
wherein the oral dosage form provides pain relief for up to 24 hours.

119. The method of claim 118, wherein the opioid agonist comprises oxycodone or a pharmaceutically acceptable salt thereof.

120. The method of claim 119, wherein the oxycodone or pharmaceutically acceptable salt thereof comprises between about 10 and 400 mgs of the oxycodone or pharmaceutically acceptable salt thereof.

121. The method of claim 118, wherein the oral dosage form further comprises:
at least one second opioid agonist loaded pellet or granule, wherein the at least one second opioid agonist comprises an opioid agonist or a pharmaceutically acceptable salt thereof, said at least one second opioid agonist loaded pellet or granule comprising an extended release layer, wherein the extended release layer comprises a second suitable amount of a water-insoluble polymer, said second suitable amount of water-insoluble polymer being different than the first suitable amount of water-insoluble polymer.

122. The method of claim 121, wherein the oral dosage form further comprises: at least one third opioid agonist loaded pellet or granule to the oral dosage form, wherein the at least one third opioid agonist comprises an opioid agonist or a pharmaceutically acceptable salt thereof, said at least one third opioid agonist loaded pellet or granule comprising an extended release layer, wherein the extended release layer comprises a third suitable amount of a water-insoluble polymer, said third suitable amount of water-insoluble polymer being different than the first suitable amount of water-insoluble polymer and different than the second suitable amount of water-insoluble polymer.

123. The method of claim 118, wherein the oral dosage form further comprises:
an opioid antagonist loaded pellet or granule, wherein the opioid antagonist layer comprises an opioid antagonist or a pharmaceutically acceptable salt.

124. The method of claim 123, wherein the opioid antagonist is selected from the group consisting of naltrexone, naloxone, nalmephene and nalorphine.

125. The method of claim 118, wherein the oral dosage form further comprises: a drug loaded pellet or granule, wherein the drug loaded pellet or granule is loaded with at least one pain management drug or at least one non-steroidal anti-inflammatory drug (NSAID).

126. The method of claim 125, wherein the pain management drug is selected from the group consisting of oxycodone, codeine, morphine, hydromorphine, anilevidine, merperidine, methadone, levorphanol, pentazocine, propoxyphene, alfentanil, allyprodine, alphaprodine, anileridine, benzylmorphine, bezitramide, buprenorphine, butorphanol, clonitazene, cyclazocine, desomorphine, dextromoramide, dezocine, diampromide, dihydrocodeine, dihydromorphine, dimenoxadol, dimepheptanol, dimethylthiambutene, dioxaphetyl butyrate, dipipanone, eptazocine, ethoheptazine, ethylmethylthiambutene, ethylmorphine, etonitazene, fentanyl, heroin, hydrocodone, hydromorphone, hydroxypethidine, isomethadone, ketobemidone, levallorphan, levophenacylmorphan, lofentanil, meptazinol, metazocine, metopon, myrophine, nalbuphine, narceine, nicomorphine, norlevorphanol, normethadone, nalorphine, normorphine, norpipanone, opium, oxymorphone, papavretum, phenadoxone, phenomorphan, phenazocine, phenoperidine, piminodine, piritramide, propheptazine, promedol, properidine, propiram, sufentanil, tramadol, tilidine, salts thereof, and combinations thereof; and
wherein the NSAID is selected from the group consisting of diclofenac, flufenamic acid, flurbiprofen, ibuprofen, indomethacin, ketoprofen, naproxen, phenylbutazone, sulindac, piroxicam, salicylic acid, acetylsalicylic acid, celecoxib, etodolac, fenoprofen, ketoralac, oxaprozin, nabumetone, tolmetin, and rofecoxib.

127. The method of claim 118, wherein the oral dosage form further comprises:
at least one second opioid agonist loaded pellet or granule, wherein the at least one second opioid agonist comprises an opioid agonist or a pharmaceutically acceptable salt thereof.

128. The method of claim 127, wherein the opioid agonist comprises oxycodone or a pharmaceutically acceptable salt thereof.

129. The oral dosage form of claim 128, wherein the oxycodone or pharmaceutically acceptable salt thereof comprises between about 10 and 400 mgs of the oxycodone or pharmaceutically acceptable salt thereof.

130. The method of claim 118, wherein the water-insoluble polymer is selected from the group consisting of alkylcellulose, an acrylic acid polymer, an acrylic acid copolymer, a methacrylic acid polymer, a methacrylic acid copolymer, shellac, zein, and hydrogenated vegetable oil.

131. The method of claim 130, wherein the water-insoluble polymer comprises Eudragit NE 30D.

132. The method of claim 118, wherein the extended release layer further comprises a lubricant.

133. The method of claim 132, wherein the lubricant is selected from the group consisting of calcium stearate, magnesium stearate, zinc stearate, stearic acid, talc and a combination thereof.

134. The method of claim 118, wherein the at least one opioid agonist-loaded pellet or granule is further coated with a sealing layer.

135. The method of claim 118, wherein the opioid agonist layer further comprises a binder agent.

136. The method of claim 135, wherein the binder agent is selected from the group consisting of hydroxypropylmethyl cellulose, hydroxyethyl cellulose, hydroxypropyl cellulose, methyl cellulose and polyvinyl pyrrolidone.

137. The method of claim 118, wherein the oral dosage form is in a capsule or granule form.

138. The method of claim 118, wherein the subject has osteoarthritis or rheumatoid arthritis.

139. The method of claim 118, wherein the oral dosage form is administered to the subject once every 24 hours.
